(19) United States
(12) Patent Application Publication
(10) Pub. No.: US 2004/0010131 A1
(43) Pub. Date: Jan. 15, 2004

(54) Xanthomonas genes associated with plant pathogenicity

(76) Inventors: Ana Claudia Rasera da Silva, Sao Paulo (BR); Shaker Chuck Farah, Sao Paulo (BR); Ronaldo Bento Quaggio, Sao Paulo (BR); Fernando de Castro Reinach, Sao Paulo (BR); Jesus Aparecido Ferro, Jaboticabal (BR); Julio Cezar Franco de Oliveiro, Jaboticabal (BR); Marcelo Luiz de Laia, Jaboticabal (BR); Joao C. Setubal, Campinas (BR); Luiz Roberto Furlan, Botucatu (BR)

Correspondence Address: FULBRIGHT & JAWORSKI, LLP, 666 FIFTH AVE, NEW YORK, NY, 10103-3198, US

(21) Appl. No.: 10/418,861

(22) Filed: Apr. 17, 2003

Related U.S. Application Data
(60) Provisional application No. 60/374,620, filed on Apr. 22, 2002.

(51) Int. Cl. C07H 21/02; C07H 21/04

(52) U.S. Cl. 536/23.1

(57) ABSTRACT

Isolated nucleic acid molecules which are characteristic of pathogenic strains of Xanthomonas are described, as are variants. The variants demonstrate modified virulence as compared to reference sequences, and permit one to connect virulence with specific portions of the genome of Xanthomonas. Proteins encoded by these sequences, as well as other aspects of the invention including recombinant cells, expression vectors, and diagnostic assays, are also disclosed.

### RELATED APPLICATION

This application is a continuation in part of Ser. No. 60/374,620, filed Apr. 22, 2002, incorporated by reference in its entirety.

### FIELD OF THE INVENTION

This invention relates to isolated nucleic acid molecules of various Xanthomonas species, as well as their use in various methods and approaches to, e.g., plant pathology, such as by identifying nucleic acid molecules and proteins involved in pathology caused by the bacterial pest.

### BACKGROUND AND PRIOR ART

The bacterial genus “Xanthomonas” comprises a group of bacterial pathogens which show remarkable diversity and which have great economic importance. Two members of this genus, which are the subject of this patent application, are “X. axonopodis pv. citri” (“Xac” hereafter), and “X.campestris pv. campestris” (“Xcc ” hereafter). The two bacteria have distinct host ranges and disease phenotypes. For example, “Xac” is the causal agent of citrus canker, which is a serious disease which affects citrus cultivars, resulting in significant, worldwide economic loss. The symptoms of the disease are well known to the skilled artisan, and include canker lesions on fruit, leaves and stems, leading to abscission of fruit and leaves, twig dieback and general decline of the plant. When the citrus leaf miner is present, infection is dramatically enhanced. See, e.g., Gottwald, et al., “Compendium of Citrus Diseases” in Timner, et al., ed Compendium of Citrus Diseases, Am. Phytopathological Society (St. Paul, 2000). Disease control is limited to prevention, and drastic, large scale rouging of trees.

Xcc, in contrast, causes black rot, a disease of crucifers, including Brassica and Arabidopsis. It is a vascular pathogen which invades xylem, and colonizes mesophyll. Among the symptoms are leaf chlorosis, necrosis and darkening of leaf veins and vascular tissue within the stem. Extensive yellowing, wilting, and necrosis occur with advancing disease. See Hayward, et al., “The Host of Xanthomonas” in Swings, et al., Xanthomonas, Chapman & Hall (1993). Commercially, Xcc is grown to produce xanthan gum, which is a well known vicosifying and stabilizing agent.

It would be useful to have means available for identifying particular Xanthomonas species, “Xac” and “Xcc,” as defined above, in particular. Further, it would be useful to have materials available which could be used to distinguish between the bacteria, and to be used in, e.g., developing mechanisms for addressing infections and infestations of the bacteria.

The tools available to the molecular biologist are many, and powerful. The advances that have been witnessed in the fields of bioinformatics and genomics over the past decade are well known and need not be reiterated here.

These techniques have been applied to both Xac and Xcc, to determine the complete genomes of these organisms. Further, these data have been utilized to determine what molecules are unique to each organism, vis a vis each other and to prokaryotes as a whole. These, and other feature of the invention will be elaborated upon in the disclosure which follows.

### DETAILED DESCRIPTION OF PREFERRED EMBODIMENTS.

### EXAMPLE 1

The bacteria used were Xanthomonas axonopodis pv citri (strain 306), and Xanthomonas campestris pv campestris (strain ATCC 333913). The “Xac” species is known to have a single chromosome and two plasmids, while the “Xcc” species contains a single chromosome. The genomes were sequenced via shotgun methodologies, in accordance with Fleischmann, et al., Science 269:496-512 (1995), incorporated by reference. To elaborate, several pUC18 libraries were generated, containing inserts ranging from 1 to 4 kilobases in size. A total of 205, 408 “reads” from Xac were sequenced, from both ends, and the comparable number for Xcc was 174,051. In addition, several cosmid libraries were generated, with inserts ranging from 30 to 45 kilobases in size. A total of 7380 Xac cosmids, and 5476 Xcc cosmids were sequenced from both ends. Assembly was then carried out, using the phred/phrap/consed package, described by Ewing, et al, Genome Res. 8:186-194 (1998); Ewing, et al, Genome Res. 8:175-185 (1998); and Gordon, et al., Genome Res. 8:195-202 (1998), all of which are incorporated by reference, as well as a scaffolding program. Assembly was then confirmed, by comparing computational prediction to CeuI, SwaI and PmeI restriction maps.

The assembly resulted in a total of 103 virtual gaps in Xac, and 135 in Xcc, many of which were a result of the rapid screening process which was required for scaffolding. The gaps were closed by sequencing whole plasmids (20 for Xac, and 67 for Xcc), and whole cosmids (49 for Xac, 13 for Xcc). The overall estimated error rate for the Xac sequence is less than 1 in 10,000 base pairs, with no base pair having phred error quality below 20. With respect to the Xcc sequence, there was a total of 15 uncertain bases, due to resolution errors within regions of secondary structure.

### EXAMPLE 2

Following the sequencing work described supra, the genomes were analyzed. Xac has one, circular chromosome containing 5, 175, 554 base pairs, and two plasmids referred to as pXAC33 and pXAC 64, which contain, respectively, 33,699 and 64,920 base pairs. Xcc has one, circular chromosome containing 5,076,187 base pairs. The nucleotide sequences are presented in Ser. No. 60/374,620 and are incorporated by reference in their entirety.

Summaries of the analysis of the genomes are presented in tables 1A-1C, attached hereto, with details presented infra.

The chromosomes were aligned, to determine the degree of alignment. It was observed that the alignment was not perfect, and three rearrangement events are believed to be responsible for this. The first is an inversion around the proposed terminus of replication. Second and third are different sized inversions around the origin of replication.

On the protein level, 2,929 collinear genes were paired corresponding to 70.0% and 67.9%) of the Xcc and Xac proteomes. There were an additional 512 non-collinear, or translocated, pairs of orthologous genes. Further, Xcc contains 646 genes (15.4%) not found in Xac, and Xac contains 800 (18.5%) genes not found in Xcc. Approximately 40% of the strain specific genes can be assigned specific functions, as set forth in table 1A.

Xac and Xcc show homology with Xyllela fastidiosa. Based upon the homology therebetween, the origin of replication for both the Xac and Xcc chromosomes were positioned in a region between the 50S ribosomal protein L34, and the gvrB genes, a placement consistent with the GC-skew inversion, discussed infra.

In both genomes, the large inversion around the putative terminus of replication contains a large number of strain specific genes, some of which are clearly involved in pathogenesis. In Xac these genes include: (a) syrE related genes, similar to those responsible for the synthesis of the fungicide syringomycin in Pseudomonas syringae; (b) and insertion of 133,025 bp that harbors many genes not commonly found in γ-proteobacteria, as well as 19 genes also found in Xylella fastidiosa CVC, including a putative RTX like—toxin; (c) nodV/nodW two-component system homologs, which in Bradyrhizobium japonicum respond to plant-derived isoflavonone signals and participate in the alternative regulatory pathway for nodulation. In Xcc, the genes include: (a) two copies of *1f filamentous phage, specific to X.campestris pv. campestris strains; (b) an additional cyclic beta 1,2 glucan synthetase, associated with virulence/colonization in members of the Rhizobiaceae family (Agrobacterium, Rhizobium and Brucella); (c) an avirulence gene similar to avrBs1; (d) a tannase, that allows microorganisms to overcome growth inhibition induced by plant tannins; (e) four genes similar to those involved in antibiotic synthesis in Streptomyces (macrolides and streptomycin); and (f) a cluster of genes related to nitrate assimilation. These observations suggest that a significant proportion of the strain-specific genes involved in pathogenicity were acquired through recombination events in this region.

The two plasmids found in Xac, and summarized in Table 1c, harbor four copies of pthA genes (two per plasmid), which code for AvBs3-type avirulence proteins. In each plasmid, one of the pthA genes is flanked by a transposon (Tn5045), which shares 76% nucleotide identity with the transposon Tn5044, described by Tu, et al., Mol. Gen Genet. 217:505-510 (1989) except that it does not carry the mercury resistance operon. All copies of pthA share a conserved sequence (gccatcgccagca) with Tn5045 that may be a substrate for homologous recombination. Several genes involved in plasmid stability are present: kor/kfr, parA/parB/parC and pemI/pemK. Members of the kor/kfr pair are located in distinct plasmids and may be working in trans. Three copies of the repA gene, involved in plasmid replication, were found, two in pXAC33 and one in pXAC64. The conjugation genes trwB and trwC and the virB operon were found in pXAC64.

The genomes of both organisms are rich in transposable elements (TE), either as insertion sequences (IS) or transposons (Tn). A total of 109 genes in Xcc and 108 genes in Xac related to 27 types of TE, were identified, only four of which are common to both genomes. In Xcc, the IS5-family is highly represented with 16 copies of IS1478 (Chen , et al., J Bacteriol 1818:1220-1228 (1999)) while in Xac, the IS3-family is more abundant with 21 copies of ISxac3. Many of these elements are located near the insertion events described supra.

Phylogenetic analyses place both Xac and Xcc at the base of the γ- protobacteria subdivision, in the same clade as X. fastidiosa. The divergence time between Xac and Xcc is estimated at 6.5 to 8.9 million years. For comparison, the estimated divergence time between Xac and X. fastidiosa is 228 million years. A comparison of the predicted Xac proteome against other completed proteomes using the COG database (Tatusov, et al., Nucl. Acids Res. 29:22-28 (2001), incorporated by reference) shows that the eight organisms with the greatest number of shared COGs are either γ- or α-proteobacteria. Further analysis based on best protein sequence hits indicates that approximately 40% of all genes found in Xac or Xcc are most similar to genes belonging to organisms that are not γ-proteobacteria. Among these genes, a high proportion belongs to the α-proteobacteria. Given that three of these bacteria (Mesorhizobium loti, Sinorhizobium meliloti and Agrobacterium tumefaciens) have plants as hosts, it is thought that many of the shared genes may be specific to and important in plant-bacteria interactions.

The Xanthomonas species present a large and diversified set of metabolic pathways. Both genomes contain a complete set of genes predicted to encode enzymes for the utilization of glucose, glycerol and disaccharides. The pentose phosphate and gluconeogenesis pathways are complete. All of the other 18 amino acids can be degraded via the TCA cycle with the exception of tryptophan and lysine. The beta-oxidation pathways and the glyoxylate cycle are also complete, and provide both bacteria with the capacity to generate glucose from fatty acids.

Xcc harbors a cluster of 7 genes (cysG, nasTACDEF) required to assimilate and convert nitrate and nitrite into ammonium (Lim, et al., J Bacteriol 176:255-2559 (1994)). None of these are found in Xac; rather, the presence of an ABC-type oligopeptide transport system (oppA, oppB andoppC) was detected, which may facilitate the entry of small oligopeptide products to be used as a nitrogen source. Xac also contains additional putative genes related to proteases, including three proteins similar to secreted xanthomonapepsins/pseudomonapepsins. The capacity to assimilate nitrates may enhance the ability of Xcc to survive in the relatively nutrient-poor xylem, where nitrate is the major form of nitrogen.

Xac and Xcc have an extensive repertoire of proteins for cell wall degradation. Both genomes encode enzymes with cellulolytic, pectinolytic and hemicellulolytic activities. Xcc has more genes involved in pectin and cellulose degradation that Xac. Xcc has two 1,4 beta-cellobiosidases and two pectin esterases not present in Xac. The lack of these enzymes does not preclude the degradation of cellulose and pectin by Xac. These differences may denote and adaptation to degrade host cell wall components and could account for the massive degeneration of plant tissue observed in black rot disease, while citrus canker caused by Xac shows mainly local tissue necrosis.

Both genomes contain a large number of genes involved in the synthesis of small molecules. As with E. coli and other prototrophic bacteria, Xcc and Xac harbor all the genes necessary for the synthesis of amino acids from chorismate, pyruvate, 3-phosphoglycerate, glutamate and oxaloacetic acid. The pathways for the synthesis of nucleotides, purines and pyrimidines are complete. Moreover, both Xanthomonas harbor at least 50 genes similar to those involved in the synthesis and elongation of fatty acids from acetate. The bacteria produce an extensive variety of enzyme cofactors and prosthetic groups. In contrast to X. fastidiosa, both organisms are able to synthesize pyrrolquinoline quinone, a non-covalently bound cofactor required by quinoproteins such as glucose and methanol/ethanol dehydrogenases. (Anthony, et al., Prog. Biophys. Mol. Biol. 69:1-21 (1998)). Xac and Xcc are predicted to code for approximately 270 small-molecule transporters (6% of all genes). Both xanthomonas also produce membrane-bound brominated aryl-polyene yellow pigments, known as xanthomonadins, which are useful as a diagnostic, chemotaxonomic marker and may have an adaptative role in epiphytic survival (Poplawsky, et al., Appl. Environ Microbiol 66:5123-5127 (2000)). The genes involved in xanthomonadin production in both Xac and Xcc are organized in a single 5.8 kbp operon, which shows significant conservation with the corresponding cluster in X, oryzae pv oryzae (GenBank AY010120).

Most of the genes responsible for DNA synthesis, restriction and modification, repair and recombination found in E. coli are found in both genomes. The inducible DNA polymerase IV (dinP) and photolyase-like genex (Phr/Cry), absent in X. fastidiosa are found in both genomes, probably because these bacteria inhabit widely diverse environments. A number of genes involved in DNA metabolism are duplicated in both Xanthomonas, including DNA ligases, Nudix family related proteins, the main subunit of DNA polymerase III, LexA, subunits of the exonuclease ABC repair system (uvrA and uvrC), exonuclease III, and a long DNA helicase (1 hr). Duplication of uvrA, among others, has been described as one of the possible reasons for significant resistance to DNA damage observed in D. radiodurans (White, et al., Science 286:1571-1577 (1989)). The restriction/modification system of Xac has one cluster for a Type I and another for a Type II systems while Xcc has two clusters for a Type I system. These features may be linked to differences in mobile genetic elements present in each genome.

It is known that Xanthomonas has a polar flagellum and that flagella may be important for virulence; however the genes responsible for their synthesis have not been completely described until now. Chemotaxis receptors and flagella biogenesis genes are organized into four clusters spread over 150 kbp in both Xanthomonas genomes. Cluster 1 is composed of cheBDR, several methyl-accepting chemotaxis protein genes (mcp), and cheAYW. There are ten copies of the mcp genes in Xac and eight copies in Xcc, both of which are organized in tandem. Another 10 and 11 mcp copies are found scattered throughout the Xac and Xcc genomes, respectively. In Xcc there is an extra copy of cheW within the mcp repeats. Cluster 1 has a structure similar to the corresponding cluster in Pseudomonas aeruginosa described by Stover, et al, Nature 406:959-961 (2000), but the latter has fewer mcp genes organized in tandem at this position. Cluster 2 is composed of cheAZY, fliA, fleN and flhFAB, with a structure similar to that found in P. aeruginosa and recently in X. oryae pv oryae. Clusters 3 and 4 are composed of 14 (fliRQPONMLKJIHGFE) and 17 genes (fliSDC,flgLKJHGFEDCB, cheV,flgAM), respectively.

Type IV fimbriae (pili) are key structures for host colonization and adhesion in pathogenic bacteria. Xac has 25 genes involved in type-IV fimbriae assembly, most of which are present in Xcc, with the exception of the pilY1 gene which is disrupted by an IS1478 insertion. In Pseudomonas aeruginosa the disruption of this gene impairs pili assembly, though the function of fimbriae in this Xcc strain needs to be determined experimentally. Fimbrillin of Xac is similar to fimA found in other xanthomonads while the collinear counterpart in Xcc is more similar to pilE from Neisseria. This fact may be related to specific differences in the interaction of these two strains with their respective hosts, since fimbrillin has been shown to be important for adhesion in pathogens. See, e.g., Kennan, et al., J Bacteriol 183:4451-4458 (2001).

Xac and Xcc have two classes of glycine-rich outer membrane proteins associated with adhesion: the filamentous hemagglutinins and the auto-transporter proteins. Filamentous hemagglutinins similar to those of Bordatella pertussis (fhaBC) and an auto-transporter YapH like protein of Yersinia pestis were found in the genomes. While the above genes were also observed in the phytopathogenic Xylella fastidiosa, their occurrence in bacteria is historically associated with adherence to epithelial tissue in mammalian diseases. Both genomes present copies (two in Xac, one in Xcc) of XadA, an outer membrane protein which has been shown to be involved in the virulence of X.oryzae pv oryzae (GenBank GI:9864182). Adhesion and protection from plant metabolites can also be mediated by exopolysaccarides (EPS) (Katzen et al, J Bacteriol 180:1607-1617 (1998)). Both Xanthomonas genomes present all genes necessary for xanthan gum biosynthesis, as previously described for other Xcc strains (Katzen, et al, J. Bacteriol 178:4313-4318 (1996)).

Distinct gene clusters responsible for O-antigen synthesis are present in Xac and Xcc. They are located in different regions of the chromosomes and lack significant sequence similarity. This is consistent with the observation that LPS O-antigen is pathovar/strain specific and may be involved in host range selection and pathogenicity, where it is predicted to function as a barrier against plaint toxins. (Dow, et al., Mal. Plant Microbe Interact 8:768-777 (1995)). In Xcc, these genes are organized in a similar manner to that previously reported by Vorholter, et al., Mol Genet. Genomics 266:79-95 (2001) while in Xac, they are divided into two regions, one containing transferases, epimerases, translocases and derived sugar transport proteins and another containing xanAB and rmlDABC genes.

The four most common systems associated with protein secretion in Gram-negative bacteria were found in both genomes and had a similar organization. The sec-independent type I system comprises several ABC transporters (67 genes in Xac and 56 genes in Xcc) and others genes involved in the export of bacterial virulence factors such as proteases and toxins. Two of these genes, hlyD and hlyB, associated with the secretion of hemolysis in E. coli, are only present in Xac, adjacent to RTX-like toxin gene.

The type II secretory system, a widely conserved sec-dependent mechanism, is involved in the extracellular secretion of degradative enzymes and toxins. Both Xanthomonas present two different type II secretion systems, one of which is the xps cluster described by Dums, et al., Mol Gen. Genet. 299:347-364 (1991). The second newly identified cluster, is most similar to that found in Caulobacter crescentus and was named “xcs.” No differences are detected among the two xanthomonads regarding the number and organization of these genes. These clusters are associated with the rearrangements described supra.

The type III secretion system (hrp pathway) is critical for pathogenicity and initiation of disease. A common cluster composed of 26 genes extending from hpa2 to hrpF is found in each genome. However, the cluster is located in different positions in the respective genomes. In both bacteria, the gene order from hrcC to hrpf is identical to the hrp pathways of X.campestris pv. vesicatoria and X. oryzae pv. oryzae. HrpW, first identified in Erwinia and Pseudomonas by Charkowski, et al. J Bacteriol 180:5211-5217 (1998), is found between hrpF and hrpE in Xcc suggesting the hrpW is part of the Xanthomonas type III system. Although hrpW is also present in Xac, it is not associated with the hrp gene cluster. Expression of the type III structural genes is typically induced in the presence of the plant host and is mediated by the hrpX and hrpG gene products, both of which are present in Xac and Xcc. The homology between the respective proteins in Xac and Xcc is high (>80% on average) with the exception of HpaP, Hpa, HpaA, HrpE and HrpW. It may be significant that some of these latter proteins are extracellular or secreted components of the type III secretion system. Therefore strain-specific differences in these components may reflect strain-specific interactions with their host.

The type IV secretion systems have been well characterized in organism such as Agrobacterium tumefaciens where the virB operon is required for the transfer of the T-DNA to the plant cell, as per Bums , et al, Curr. Opin. Microbiol 2:25-29 (1999). Other bacteria utilize this system for conjugal transfer as well as secretion of toxins or other proteins Burns, et al., supra. This is the first report of a type IV system in any species of Xanthomonas. Xac has two type IV systems, one on the chromosome and another inpXAC64. These clusters have a different organization from the Agrobacterium virB operon. VirB5 and virB7 are missing from both clusters and virD4 is missing in the plasmid. The organization of the cluster on the plasmid is very similar to that observed in Xylella fastidiosa. Xcc has only one virB cluster and the organization is similar to that on the Xac chromosome, with the exception that virB6 is translocated.

The synthesis of extracellular degrading enzymes and EPS is transcriptionally regulated by products of the rpf gene cluster. The Xcc cluster has an organization similar to that previously described of other Xanthomonas strains (rpfABFCHGDIE) (See Tang, et al., Mol. Gen Genet 226:409-417 (1991); Dow, et al., Micrbiol. 46:885-891 (2000). Xac lacks rpfH and rpfI, as does the corresponding cluster in X. fastidiosa (Dow, et al., Yeast 17:263:271 (2000)) RpfH, RpfC and RpfG are predicted to operate in a signal transduction system that couples the synthesis of pathogenicity factors to the sensing of environmental signals; however disruption of the rpfH gene does not affect the synthesis of known pathogenicity factors. Slater, et al., Mol. Microbiol. 38:986:1003 (2000). The expression levels of proteases and endoglucanases are reduced when the rpfI gene is inactivated in Xcc (Dow , et al., Microbiol 146:885-891 (2000)) suggesting that rpfI may play a part in the massive tissue degeneration observed in Xcc infection. The corresponding locus in Xac is occupied by a truncated copy of an insertion sequence.

One of the most important events in a plant bacteria interaction is the induction of genes that allow colonization. In Xanthomonas, some of the genes whose expression is activated upon contact with the host (and therefore encoding “effector proteins”) posses a consensus nucleotide sequence in their promoters. These PIP boxes, for “Plant Induced Promoters” have a sequence (TTCG . . . N16 . . . TTCGn) that is highly correlated with the regulation of gene expression by hrpX and may represent a DNA binding site for the hrpX gene product. Fenselau, et al., Mol Plant Microbe Interact 8:845-854 (1995). The genomic sequences of Xcc and Xac contain 121 and 182 PIP, respectively. A subset of 21 and 24 PIP sequences in Xcc and Xac, respectively, are found between 60 and 300 bp upstream of predicted initiation codons (Table 2). Among these, 12 are common to both genomes, of which five belong to the hrp gene cluster. The remaining seven are scattered on the chromosome, including three that have a N-terminal type II signal peptide sequence and are similar to cell wall degrading enzymes. Seven other genes that harbor PIP boxes are found only in Xcc, of which two are proteases. In Xac an additional set of ten genes including an iron receptor have PIP boxes.

Both genomes encode homologs of the effector proteins AvrBs2, originally identified in X. campestris pv. vesicatoria (Kearney, et al., Nature 346:385-6 (1990)) and AvrPphE, orginally identified in Pseudomonas syringae pv. phaseolicola (Manfield, et al., Mol Plant Microbe Interact 7:726-739 (1994). These genes have PIP boxes and belong to a class of avirulence effector proteins which are fundamental to the development or restriction of disease. Table 3 summarizes all known avr genes found in these sequenced genomes. Comparative analyses indicate that Xcc contains a more diverse arsenal of known avirulence proteins, including members of the AvrBs1, AvrC, YopJ and AvrXca families although no members of the AvrBs3 family were identified. YopJ and related proteins have been proposed to function as SUMO proteases (Orth, et al., Science 290:1594:1597 (2000)) while little is known about the functions of other avr family members. Interestingly, most of the avr genes are found adjacent to transposases. The Xac genome contains four copies of the avrBs3/pthA gene family, which has been demonstrated to induce tissue hypertrophy in plant cell culture and hypersensitive response in non-host plants. These proteins are characterized by the presence of multiple 34 amino acid repeats. The two genes in pXAC64 contain 17.5 and 15.5. repeats, while the genes in pXAC33 have 16.5 and 15.5 repeats. None of the genes are absolutely identical to the previously described pthA and do not have associated PIP-boxes.

Both Xac and Xcc also contain genes related to the leucine rich repeat (LRR) proteins commonly involved in protein/protein interactions (Table 3). These LRR motifs are found in the three major classes of plant resistance genes and in the PopC protein of Ralstonia solanacearum, as shown by Young, Curr. Opin. Plast Biol 3:285-290 (2000); Giveneron, et al., Mol Microbiol 36:261-277 (2000)). The presence of distinct LRR proteins in these genomes, under control of putative PIP-Boxes suggests that they may play an important role in some stage of plant colonization.

### EXAMPLE 3

These experiments were designed to determine which genes are involved in the pathogenic process by which Xac attacks a plant host.

Samples of Xac were treated to create disruptions in their genome, with commercially available, Tn5 tranposon-transposase complex. The commercially available complex confers kanamycin resistance when incorporated into a genome.

Individual transposons were identified by culturing samples of Xac in 96 well plates containing 200 μl of TSA medium per well, supplemented with 100 μl/ml of kanamycin, and then grown for two days, at 28° C.

As a result of this, a library of approximately 10⁴ mutants was obtained, and stored at −80° C.

To determine which specific mutations impacted virulence, mutants were individually inoculated into susceptible lemon host plants, and plants were then selected for those exhibiting defects in pathogenicity. In brief, suspensions of the mutants were prepared, at 10⁸ cfu/ml, in sterile tap water, and then were pressure infiltrated into lemon leaves. Symptoms were analyzed 2-15 days after inoculation.

About 2000 mutants were analyzed, and approximately 44 were found to show altered pathogenicity phenotypes. Some of the mutants failed to induce disease symptoms at all, while others showed reduced symptoms of citrus canker.

To elaborate on this, early symptoms of citrus canker appear 2-3 days after inoculation, appearing as water soaked looking circular spots covering the inoculated area. At 3-7 days post inoculation, the pathogen induces unsure hyperplasea, with raised lesions showing elevated margins and sunken centers. In a third stage, the lesions become necrotic, and darken into a brown, corky canker.

Mutated genes which did not exhibit the complete pathogenic pathway described above are identified in the table below. In order to identify the insertion sites, the border of the kanamycin resistance gene was sequenced, toward chromosomal sequences. Terminology employed follows the table. Within the listing, “Old SEQ ID NO:” refers to the SEQ ID NOS: of the nucleotide and amino acid sequences in the provisional application, and “New SEQ ID NO:” refers to those employed herein. The sequence names remain the same.

| Sequence | Old | | |
|---|---|---|---|
| Name | SEQ ID NO: | New SEQ ID NO: | Phenotype* |
| XAC0798 | 1589, 1590 | 1, 2 | hyp 0, nec |
| XAC1927 | 3795, 3796 | 3, 4 | hyp 0, nec |
| XAC3136 | 6179, 6180 | 5, 6 | hyp 0, nec |
| XAC2053 | 4074, 4048 | 7, 8 | hyp 0, nec |
| XAC3457 | 6817, 6818 | 9, 10 | hyp 0, nec |
| XAC2639 | 5189, 5190 | 11, 12 | hyp 0, nec |
| XAC2047 | 4035, 4036 | 13, 14 | hyp 0, nec |
| XAC1201 | 2363, 2364 | 15, 16 | hyp 0, nec |
| XAC0144 | 287, 288 | 17, 18 | hyp 0, nec |
| XAC3320 | 6543, 6544 | 19, 20 | hyp 0, nec |
| XAC2636 | 5783, 5784 | 21, 22 | hyp 0, nec |
| XAC4040 | 7967, 7968 | 23, 24 | Absense of symptoms |
| XAC0789 | 1571, 1572 | 25, 26 | Absense of symptoms |
| XAC0340 | 679, 680 | 27, 28 | Absense of symptoms |
| XAC3839 | 7579, 7580 | 29, 30 | Absense of symptoms |
| XAC3673 | 7249, 7250 | 31, 32 | Absence of symptoms |
| XAC3980 | 7849, 7850 | 33, 34 | Absence of symptoms |
| XAC0410 | 817, 818 | 35, 36 | Absence of symptoms |
| XAC0356 | 711, 712 | 37, 38 | hyp -, nec |
| XAC3607 | 7117, 7118 | 39, 40 | hyp -, nec |
| XAC1669 | 3289, 3290 | 41, 42 | hyp -, nec |
| XAC0756 | 1505, 1506 | 43, 44 | hyp -, nec |
| XAC3285 | 6475, 6476 | 45, 46 | hyp -, nec |
| XAC3294 | 6493, 6494 | 47, 48 | hyp -, nec |
| XACb0024 | 131, 132 | 49, 50 | hyp -, nec |
| XAC2118 | 4159, 4160 | 51, 52 | hyp -, nec |
| XAC4291 | | 53 | hyp -, nec |
| XAC3245 | 6395, 6396 | 54, 55 | hyp -, nec |
| XAC0095 | 189, 190 | 56, 57 | hyp -, nec |
| XAC0014 | 27, 28 | 58, 59 | hyp -, nec |
| XACb0015 | 113, 144 | 60, 61 | hyp -, nec |
| XAC2126 | 4175, 4176 | 62, 63 | hyp -, nec |
| XACb0067 | 217, 218 | 64, 65 | light tan canker |
| XAC0102 | 203, 204 | 66, 67 | light tan canker |
| XAC0618 | 1229, 1230 | 68, 69 | ws 0, hyp-, nec 0 |
| XAC2616 | 5147, 5148 | 70, 71 | ws 0, hyp-, nec 0 |
| XAC1495 | 2947, 2948 | 72, 73 | ws 0, hyp-, nec 0 |
| XAC3704 | 7311, 7312 | 74, 75 | ws 0, hyp-, nec 0 |
| XAC4160 | 8207, 8208 | 76, 77 | ws 0, hyp-, nec 0 |
| XAC3225 | 6355, 6356 | 78, 79 | hyp -, nec - |
| XAC3984 | 7857, 7858 | 80, 81 | hyp - |
| XAC3263 | 6431, 6432 | 82, 83 | hyp - |
| XAC3600 | 7103, 7104 | 84, 85 | hyp - |

*
hyp 0, nec → no hyperplasia, necrotic lesion
hyp -, nec → reduced hyperplasia, necrotic lesion
ws 0, hyp-, nec 0 → no water-soaking, reduced hyperplasia, no necrosis
hyp -, nec - → reduced hyperplasia, reduced necrosis
nec - → reduced necrosis
hyp - → reduced hyperplasia

The foregoing description sets forth various features of the invention, including isolated nucleic acid molecules which encode proteins unique to either Xac or Xcc, or which are unique to one or both of the two Xanthomonas species, relative to other bacteria and all other organisms. Many examples of such nucleic acid molecules are given in the disclosure, supra, as well be clear to the skilled artisan who reviews the information. Such isolated nucleic acid molecules can be used, e.g., diagnostically, to determine if Xac and/or Xcc is present. The artisan of ordinary skill is well familiar with such assays, such as fluorescent hybridization assays, or other formats which utilize nucleic acid molecules. Also encompassed are variants of these sequences. “Variants” as used herein refers to nucleic acid molecules which share at least 75%, preferably at least 80%, and most preferably at least 85% identity with one of the nucleic acid molecules set forth herein (SEQ ID NO: 1, 3, 5, 7, 9 . . . 53 and 54, 56 . . . 84) but with modified virulence as compared to the sequences disclosed herein. The experiments of example 2 show how to determine this.

Similarly, nucleic acid molecules can be used, e.g., to develop antisense molecules which can be used to prevent or to reduce the expression of one or more relevant genes. Such antisense molecules are useful because, as was noted, supra, the bacterial species are pathogenic and harmful to their plant hosts. Hence, the generation and use of antisense molecules presents a way to control the bacterial pathology.

The nucleic acid molecules may be used, e.g., to create expression vectors in which they are operatively linked to promoters. The resulting expression vectors can be used to transform or to transfect cells, be the eukaryotic or prokaryotic. Such cells can then be used to produce another feature of the invention, which are the proteins that are unique to Xac and/or Xcc. These proteins may be produced recombinantly, and if so produced it is to be understood that degenerate forms of the nucleic acid molecules described supra may be used, and are also a part of the invention. Such proteins can be used, e.g., to make antibodies that can then be used to detect the presence of the bacteria in samples, via any of the standard immunoassay methods known to the art. Variant proteins in accordance with the variant nucleic acid molecules are also part of the invention. The nucleic acid molecules can also be used in connection with screening methodologies to identify pre-existing or new materials useful in inhibiting disease. For example, one can test a transformed or transfected cell with the substance of interest, to determine if it has an impact on the effect of the gene on the plant.

Another aspect of the invention are computer readable media which have recorded thereon all or a part of the nucleotide sequences, and/or amino acid sequences set forth herein, or degenerate variants thereof. Exemplary of such computer readable media are floppy discs, hard discs, random access memory (RAM), read only memory (ROM), and CD-ROMs. Such computer readable media are useful, e.g., in identifying whether a nucleic acid molecule of interest is from, or is homologous to, an Xac and/or Xcc nucleic acid molecule. Thus, they permit the skilled artisan to determine if a plant, plant part, or collection of plants, such as an orchard, are infected with Xac and/or Xcc, or if an organism is potentially pathogenic to plants, based upon homology to Xac and/or Xcc.

Further, they provide a library for study of what molecules are involved in a diseased condition, e.g., one can analyze specimens taken from a stricken orchard, analyze bacterial DNA found therein, and compare it to the materials set forth as part of this invention.

Other aspects of the invention will be clear to the skilled artisan and need not be set forth herein.

The terms and expressions which have been employed herein are used as terms of description and not of limitation, and there is no intention in the use of such terms and expressions of excluding any equivalents of the features shown and described or portions thereof, it being recognized that various modifications are possible within the scope of the invention.

We claim

1. An isolated nucleic acid molecule form a Xanthomonas microorganism, wherein said isolated nucleic acid molecule is associated with pathogenicity caused by said Xanthomonas microorganism, or a variant thereof which causes reduced or enhanced pathogenicity.

2. The isolated nucleic acid molecule of claim 1, wherein said Xanthomonas microorganism is X axonopodis pv citri (Xac).

3. The isolated nucleic acid molecule of claim 1, selected from the group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 54, 56, 58, 60 . . . 84.

4. Expression vector comprising the isolated nucleic acid molecule of claim 1, operable linked to a promoter.

5. Recombinant cell, transformed or transfected with the isolated nucleic acid molecule of claim 1.

6. Method for determining if Xanthomonas bacteria is present in a sample, comprising contacting said sample with an oligonucleotide which binds specifically to the isolated nucleic acid molecule of claim 1, and determining said binding as an indication of presence of Xanthomonas in said sample.

7. An isolated protein encoded by the isolated nucleic acid molecule of claim 1.

8. The isolated protein of claim 7, selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 55, 57, 59, 61, 63, 65, 67, 69, 71, 73, 75, 77, 79, 81, 83, 85.

9. An antibody which binds specifically to the isolated protein of claim 7.

10. The antibody of claim 9, wherein said antibody is a polyclonal or a monoclonal antibody.

11. Method for determining presence of Xanthomonas in a sample comprising contacting said sample with an antibody which binds to the protein of claim 7, and determining said binding as an indication of presence of Xanthomonas in said sample.

12. Composition comprising at least one protein of claim 8, and a carrier.


SEQUENCE LISTING (85 sequences)

SEQ ID NO: 1 - DNA, 1428 bp, Xanthomonas
atgcgcaacacgcctgctcatgctccaccctcactgatacaatgcggtgtccgtctcttgctgactgcattggtcctgctggtgaccaccgccaacgcgcaggccgatgtcatcctgcacgccttcaactggccgtatgccaccgtggaagcgcgcgccaagcagattgccgatgccggctatcgcaaggtattggtcgcccccgcgtaccgctccgaaggaagtgcctggtgggcgcgctatcaaccccaggacatccgcctgatcgacaatccgctcggcgacaccgctgcattcaagaagatggtccaggcgctggccagcaacggcgtggaaacctatgccgacatcgtgttcaaccacatggccaacgaagcggccacacgctcggacctcaattacccaggcggcgcggtcctgtcgcagtacgcggcaaaccccggccgttacgattcgctgcgcttgttcggcacgctgcagtcaaacttcctcagcgccagcgatttcggcccggcgcagtgcatcagcaactacaacgacgcttaccaggtgcgcaattaccgtatctgcggcggcggcagcgatccaggcctgccggatttgctgggcaacgactgggtggtacagcagcagcgcgcctatctgcaggcgctcaagagcatgggcgtgaccggcttccgggtggatgcggccaagcacatgacgttcgaccatctcaatcgcgtcttcgatgccggcatccgatcgggtgtgtatgtcttcggcgaagtgatcaccggcggcggtacgggcaatggcgattacgaccagttcctggcgccgtacctgcaatccacgccgcatgcggcctacgactttccgctgttcaatgcggtgcgcaacgcgttcgccattggcgccagcatgcagcagctggtggatcccgccgcgtccggacaggcattgccgggcaatcgcgccgtgacctttgcagtgacccacgacattcccaacaacgccggattccgctatgcgattctcgacccggtggatgaaacgctggcctacgcgtacctgatcggtcgcaacggcggcatgccgatgctttataccgacaacaacgaaagcggcgacaatcgctgggtcaacgcttatctacgcgacgatctgcgccgcatgatcggcttccataatggcgtgcagggcaccgacatgcaggtcttgtcgtccagctcctgccacatcctgttccgtcgcggcagcctgggcatcgtgggcatcaacaagtgcggcaacccggtcaccaccacggtgggaatgaacaacagcgtgttgtactggaatgccgattacgtggatgccttgggctcgggcaacgtagtgcgcatttccagtagttcctacacctttacgctgccggcgcgcggggcgcgcatgtggcgccgctga

SEQ ID NO: 2 - protein, 475 aa, Xanthomonas
MRNTPAHAPPSLIQCGVRLLLTALVLLVTTANAQADVILHAFNWPYATVEARAKQIADAGYRKVLVAPAYRSEGSAWWARYQPQDIRLIDNPLGDTAAFKKMVQALASNGVETYADIVFNHMANEAATRSDLNYPGGAVLSQYAANPGRYDSLRLFGTLQSNFLSASDFGPAQCISNYNDAYQVRNYRICGGGSDPGLPDLLGNDWVVQQQRAYLQALKSMGVTGFRVDAAKHMTFDHLNRVFDAGIRSGVYVFGEVITGGGTGNGDYDQFLAPYLQSTPHAAYDFPLFNAVRNAFAIGASMQQLVDPAASGQALPGNRAVTFAVTHDIPNNAGFRYAILDPVDETLAYAYLIGRNGGMPMLYTDNNESGDNRWVNAYLRDDLRRMIGFHNGVQGTDMQVLSSSSCHILFRRGSLGIVGINKCGNPVTTTVGMNNSVLYWNADYVDALGSGNVVRISSSSYTFTLPARGARMWRR

SEQ ID NO: 3 - DNA, 1179 bp, Xanthomonas
ttgctgacgcgcgatggacgctgcatcgacaccgcgcagtaccgtcaggcccagtccccgaccgcggtgctgctcaagctcaccggtacctgcaacatcgactgtgattattgttacgactacgacgccgaacggttccgcgctcagcaatctctcggcaccatccagactacgctggcgccgctgttggagcgcggtgagccgctgtcgattgccttccatggcggcgagccattgctgcggttcggtctcatccgccaggtggtgcaatggctggaaccgcaccgcgcccgcgtcggtttctcgttgcagaccaacgggacgcgcttcacgccggagattctcgactttctggagcagcacgacttttcggtcggcctgtcgctcgatggtctggatgaagcgagcaatgcattgcgccgcacgcgggggccgcggacgccgctgcagtgcatcctggcgctgatgcgcgagcgtccacagttcgtgcgtcagcgctgcggtttcctggccgttgcctcgcgtacctcggcacccgggctgccggcgtttgcgcgctggctgcaggacttcggtgtgcagggcatgtcgatcaccttcctcgacctggccggccgcggcgccgatctcgccgacgagcggctctcgcccgaacaggccgtggatctgtatgcagggctgctggagatgatccggaacggtcagctccatagcctggcgttgcgcaacctggtcgcacgcatgaacaacctgtttacactgcagtcccgcgatctttgctatcgggggccctgcggcgcggccggcgatttcctggtgctggacgcgcagggcggcaaacggacctgcgactgcgtctatcacccgtatttcgaactgaaggcaggccaggacgggggcgacgcgccggcgcgcgctgccatcgtcgcgcgtcacagctggctgcggctgcatggccaaggctgcagccgctgtgcactgttcggactgtgcggcggcacctgcgtggccaaggcactggtgcgcactggtcacgaccgtagcgtggatccggtggaatgcgcattgtcgcggtatctgtatcccgaactgctgcgcgagttcacggctgccgaacggcctttgttcgcgtactacgcgcggcatgcgcagggcgatgcgctcgccgaggcggcatga

SEQ ID NO: 4 - protein, 392 aa, Xanthomonas
LLTRDGRCIDTAQYRQAQSPTAVLLKLTGTCNIDCDYCYDYDAERFRAQQSLGTIQTTLAPLLERGEPLSIAFHGGEPLLRFGLIRQVVQWLEPHRARVGFSLQTNGTRFTPEILDFLEQHDFSVGLSLDGLDEASNALRRTRGPRTPLQCILALMRERPQFVRQRCGFLAVASRTSAPGLPAFARWLQDFGVQGMSITFLDLAGRGADLADERLSPEQAVDLYAGLLEMIRNGQLHSLALRNLVARMNNLFTLQSRDLCYRGPCGAAGDFLVLDAQGGKRTCDCVYHPYFELKAGQDGGDAPARAAIVARHSWLRLHGQGCSRCALFGLCGGTCVAKALVRTGHDRSVDPVECALSRYLYPELLREFTAAERPLFAYYARHAQGDALAEAA

SEQ ID NO: 5 - DNA, 1008 bp, Xanthomonas
atggccgagcatcaggaagacgcagcgaaatcgcgcatttacgaggccctgctacaggcaacaccggatcttttgtatgtgttcgatatccagcatcgcttcgtctacgccaatcaggcgttgttgacgatgtggggaatgcgctgggaagacgcctgcggcaaaacctgcctggaactgggctacgagccctggcatgcggccatgcacgatgaagagatcgaacaggtgatcgcaacaggccagccggtgcgcggcgaggtgccattcccgcatacgaccaagggcctgcgcatctacgactacatttttacccccgtgttcggacccgacggcaaggtcgaggcgatcgccggcgccacgcgcgacatcaccgaacacaagcagcatgagcagcatctgcagctgttgatcaacgagctcaaccaccgggtcaagaattcgttggtgatggtgcaatcgctggcgcgccaatcgttcaccaatgccggcagcctgggcgacgcacaggaaaagcttgatgcgcgcttgctggcgctgtcgcgcgcacacgacacgctgacgcgcgaaaattgggtcagcgccgacatcctggaactgacccgcgacgctgccgcgctctatgagtcgcacgacggccagcggttcacgttgcagggtgattcttgtcgccttgatccgcggcgcgcattggcgctgtcgatggcgctgcatgagctgtgtaccaacgcgctcaagcacggcgcattatccttgccagctgggaatgtactggtttcgtgggagcgcagcacacgcggcgagcaggaactgcttgaattgatctggcgggaagccggggggccgccggtgcagccacccacgcgcaaggggttcggcacgcgtcttctggagcgtggcttgaagcacgacctcgagggcgaagttgagctgtccttcgatccggctggcgtgtgtttccgtgtgtccatcccgttgccgcgcgagcatatcgagtcacggccatga

SEQ ID NO: 6 - protein, 335 aa, Xanthomonas
MAEHQEDAAKSRIYEALLQATPDLLYVFDIQHRFVYANQALLTMWGMRWEDACGKTCLELGYEPWHAAMHDEEIEQVIATGQPVRGEVPFPHTTKGLRIYDYIFTPVFGPDGKVEAIAGATRDITEHKQHEQHLQLLINELNHRVKNSLVMVQSLARQSFTNAGSLGDAQEKLDARLLALSRAHDTLTRENWVSADILELTRDAAALYESHDGQRFTLQGDSCRLDPRRALALSMALHELCTNALKHGALSLPAGNVLVSWERSTRGEQELLELIWREAGGPPVQPPTRKGFGTRLLERGLKHDLEGEVELSFDPAGVCFRVSIPLPREHIESRP

SEQ ID NO: 7 - DNA, 2361 bp, Xanthomonas
atgcacgatagccagcttgcccagcagattgctcgcaccatcgccgatgagatcggtgcccaacccgcacaggcgcacgccgccatcgccctgctcgacgagggcgccagcgtccccttcatcgcccgctaccgcaaggaagtgaccggcggcctggacgatacgcaactacgcaatctggaaatgcgcctgacctatctgcgcgagctggaagagcgccgcgcggcgatcctgtccagcattggcgagcagggcaagctcagcgacgaattgcgcaacgagatcgtggcggccgacaccaagtcgcggctggaagacctgtacctgccctacaagcccaagcgccgcacccgcgcacagatcgcgcgcgaagccgggctggagccattggccgatggcctgttggcggacccgacgcagtccccggacgtggccgcagcagccttcatcgatgccgaaaagggcgttgcagacagcaaggcggcgctggaaggcgcgcgcgccatcctgatggagcgctggggtgaagatgccgcactggtcggtgagctgcgcagctggctaggcgacaacggcgtgatccgcgcacgggtcgccgagggcaaggaagaggccggtgccaagtatcgcgattacttcgatcacgccgaaacgctggcgaagattccctcgcaccgcttgctggcgctgtttcgcgcacgccgcgaggaaatcctgtacctggatctggatccgggcagcgacgccgaggccgggcaccagtacgccgaaggccgcgtggcgcgcaatgccggtatctccaacgaaggccgtccggccgatcgctggttgctggatgcgtgccggctgacctggcgcgccaagctgcatatgcacttgctgttggacctgttcaatcaggcgcgcgagaaggccgaggccgaggcgatcgcggtgttcggcgacaatctcaaggacctgatgctggcggcgccggcaggcccgcgcgtggtgcttgggctggatccgggcatccgtaccggttgcaagatcgccgtggtcgatgccaccggcaagctggtggccaccgaaacgatttatccgcacgagccgaagcggcagtgggatcagtcgctgcagaccatcaaaaaattgtgcatgcagcacaacgtggagctgatcgcgatcggcaacggcaccgccagccgcgagaccgacaagctggccggtgaggcgattgcgctgtgcggcgcggccaaattacagaaggtggtggtgagcgaggcgggcgcatcggtgtattcggcctcggagttcgcggccaaggaatttccgaatctggatgtatcgctgcgtggtgccgtgtcgatcgcgcgccgtctgcaggatccattggccgaactggtgaagatcgagcccaaggcgatcggcgtgggccagtaccagcacgatgtggatcaataccggctggccaaggcattggaagcgcgggtggaggactgcgtcaacgcggtcggtgtgtacgtcaataccgcctcggcggcgttgttgtcgcgcgtgtccgggttgtcgggcacggtagcggaaaatatcgtgcgtcaccgcgacgacaatggcccgttcaagcgtcgcaaggacttgctcaaggtgccgcggctgggcgacaagaccttcgagcaatgcgccggtttcctgcgcattgccgatggcgacgagccgctggacgtgtcggcggtgcatccggaagcgtatccggtggtcgagcgcatcgtgagcagcaccggcaagccgatcaaggcattgctgggcgacggcagttttctgcgtggcctgaaggcggagatgttcaccgacgaccaattcggtgtgccgacggtgcgcgacatcctcaaggagctggaaaaacccggccgcgatccgcggcccgagttcaaggcggcgcagttcgccgagggcatcgaggacatcaagcacctcaaaccgggcatggtgctcgaaggtgtggtcagcaatgtggccgcgttcggtgcgttcgtcgatatcggcgtgcatcaggatggactggtgcacatctcggcgttgtcggaaaccttcgtcaaggatccgcgcgatgtggtcaaggccggcgacatcgtcaaggtcaaggtgctggaagtggatgtggcgcgaaagcgcatcgcgctgacctgccgtttgtccgatacgccgccagcggcgggtggctctgcgcaaccgcgcgacgctcgcggcagcggtccggggcagggcaggcgcgatggtggtggacgcggtcaggcgcaggccaacagcaaaccgcggatcaccgcaccgccggcagacaatgcgctggcagcggcattcgcgcgtgccaaacgtagctaa

SEQ ID NO: 8 - protein, 786 aa, Xanthomonas
MHDSQLAQQIARTIADEIGAQPAQAHAAIALLDEGASVPFIARYRKEVTGGLDDTQLRNLEMRLTYLRELEERRAAILSSIGEQGKLSDELRNEIVAADTKSRLEDLYLPYKPKRRTRAQIAREAGLEPLADGLLADPTQSPDVAAAAFIDAEKGVADSKAALEGARAILMERWGEDAALVGELRSWLGDNGVIRARVAEGKEEAGAKYRDYFDHAETLAKIPSHRLLALFRARREEILYLDLDPGSDAEAGHQYAEGRVARNAGISNEGRPADRWLLDACRLTWRAKLHMHLLLDLFNQAREKAEAEAIAVFGDNLKDLMLAAPAGPRVVLGLDPGIRTGCKIAVVDATGKLVATETIYPHEPKRQWDQSLQTIKKLCMQHNVELIAIGNGTASRETDKLAGEAIALCGAAKLQKVVVSEAGASVYSASEFAAKEFPNLDVSLRGAVSIARRLQDPLAELVKIEPKAIGVGQYQHDVDQYRLAKALEARVEDCVNAVGVYVNTASAALLSRVSGLSGTVAENIVRHRDDNGPFKRRKDLLKVPRLGDKTFEQCAGFLRIADGDEPLDVSAVHPEAYPVVERIVSSTGKPIKALLGDGSFLRGLKAEMFTDDQFGVPTVRDILKELEKPGRDPRPEFKAAQFAEGIEDIKHLKPGMVLEGVVSNVAAFGAFVDIGVHQDGLVHISALSETFVKDPRDVVKAGDIVKVKVLEVDVARKRIALTCRLSDTPPAAGGSAQPRDARGSGPGQGRRDGGGRGQAQANSKPRITAPPADNALAAAFARAKRS

SEQ ID NO: 9 - DNA, 648 bp, Xanthomonas
atgactcccttcacccgacacaccggattggtcgcgccgctggatcgcgccaatgtcgataccgaccagatcattcccaagcagttcctcaagtcgatcaagcgcaccggtttcggtccgaacctgttcgacgagtggcgctatctggatatcggcgagcctggtcgcgacaacagcacgcgcccgctcaatccggaattcgtgctcaatttcccgcgctatcagggcgccagcgtcttgctggcccgggaaaacttcggctgtggttcctcgcgcgagcatgcgccgtgggcgctggacgaatacggctttcgcaccgtgatcgcgccgagctttgccgacattttctacaacaacagtttcaagaacggcctgctgccgatcgtgctggccgaagcgcaggtcgatgcattgttcgaacaatgcctgacgaccgagggctaccagctcaccgtagatctggccgcgcaacgcgtacgccgccccgatggcgtggagtacggcttcgacatcgatgccttccgcaaacactgcctgctcaatggcctggacgatatcggcctcaccttgcaggacgccgatgccatcgggcgcttcgagcaaggccatcgcgcgcgccaaccgtggttgtttggcgcgttgcagtaa

SEQ ID NO: 10 - protein, 215 aa, Xanthomonas
MTPFTRHTGLVAPLDRANVDTDQIIPKQFLKSIKRTGFGPNLFDEWRYLDIGEPGRDNSTRPLNPEFVLNFPRYQGASVLLARENFGCGSSREHAPWALDEYGFRTVIAPSFADIFYNNSFKNGLLPIVLAEAQVDALFEQCLTTEGYQLTVDLAAQRVRRPDGVEYGFDIDAFRKHCLLNGLDDIGLTLQDADAIGRFEQGHRARQPWLFGALQ

SEQ ID NO: 11 - DNA, 702 bp, Xanthomonas
atgaaaaatcagctcctgcagggcgacgccctgaccatcctacccacgctcgaagcgaattcgttcgacgcgctgatcactgacccgccatatgccagcggtgggctgaccgccgctgcccgtgcgcggccgccctcgaccaagtactgccgggatggagggcacgccgacttcgttggcgacgaacgcgaccagcgctcgcacctgaagtggatgcacctgtggctgtccgagtgcgcgcgcgtgctcaaggacggcgcgccggtgctgctgttcaccgactggcgacagctgacgctgaccaccgacgcgctacagatcgcggggtttacctggcgcggcatcactgtctgggacaagactgaaggcgtgcggccgcagttgggccgattccgcaaccaggccgagtacatcgtgtggggcagcaagggcaacatgccgctggatcgccgcgcgccggtgctgccaggtgttatccgcgagtccgtacgcaaggctgacaagcaccacctgaccggcaagcccaccgaattgatgcggcagctggtgcggatctgcgaggcaggtgggcgcgtgctcgatccgtttgctggcagcggaaccacattggtggccgcgcagctggaaggattcgaggcggtcgggattgagatgacggatcagtacgccaccgttacccgcgaccggctgacagcgctgtaa

SEQ ID NO: 12 - protein, 233 aa, Xanthomonas
MKNQLLQGDALTILPTLEANSFDALITDPPYASGGLTAAARARPPSTKYCRDGGHADFVGDERDQRSHLKWMHLWLSECARVLKDGAPVLLFTDWRQLTLTTDALQIAGFTWRGITVWDKTEGVRPQLGRFRNQAEYIVWGSKGNMPLDRRAPVLPGVIRESVRKADKHHLTGKPTELMRQLVRICEAGGRVLDPFAGSGTTLVAAQLEGFEAVGIEMTDQYATVTRDRLTAL

SEQ ID NO: 13 - DNA, 1224 bp, Xanthomonas
atggcaagcagcggttcggacaacggcaacttcgacgacaaggcgcgccaatactgggctgcctggggagatgccatgcgccacggccaggccggcgccgctgcgcaacaaccaccgcccacgtccggtagcgacgccccgcaggattggcgcaaggccgtggattggtggtcgcaattgctgcccacccaggccgccccgcaggcgcaggaggccatcgaccgctttcgcacccaggccggcgactggtttggcaccatgcagcaggtggccgcgcagtttgccgggcgcgacacctcggccaatgaagtcgccgatgcctggcgccaggccgtacaagggcagggtgagcagctgatgcagtggacgctgggttcgctgcgcggcggcagcccgggcgggttcgacccgtggctgcaggacgcggcgcaagcgctgcaaaagtggcgcgaggaaaatgcgccttggctggacatgccggcgttcgggttgaaccgcaaccaccagtcgcgcctgcagaaactggcacgcgcgcagcaggagttccaggcccagtcggaggcctatggcgagcaactcaaggcggcgatcgagcaggcgtttgcgcgcttcgcgtccaggctgagcgagcacgagagcagcggcagccagttgaccagcgcccgcgccctgttcgatctatggatcgaagcggccgaagagtcctatgccgatgttgccttgtcggagcagttccgcaaggtgtacggcggctttgccaacgcgcacatgcgcctgcgcgctgcgctgcaggaagaggtcgagcaactcagcgaacgcttcggcatgcccacacgttcggagatggacgccgcgcatcgccgcattgccgaactggaacgcctggtcaggcggatgctgcgtaacgcagcttcgccggcgagcaagcccgctgcagcggcgcagcctgcaccggtgacaccggcagcgggcaagcgcgcgtcgtccgccggggccagcgctgttgcgcggccggccggggcagaacgggcagtcaagcagggtgcatcgaagaaggcacccgtcgacaagaccgcgacgaaaaaggcggctccgaaaaaggcgccagccgtttctaagccgacgaaaacgacctcgcccaagaagcatggtggcgcgacatcctcgcccgagcttgcggcccgcaaacccgtcgcagctaaaaaacgtggagcgcaggcatga

SEQ ID NO: 14 - protein, 407 aa, Xanthomonas
MASSGSDNGNFDDKARQYWAAWGDAMRHGQAGAAAQQPPPTSGSDAPQDWRKAVDWWSQLLPTQAAPQAQEAIDRFRTQAGDWFGTMQQVAAQFAGRDTSANEVADAWRQAVQGQGEQLMQWTLGSLRGGSPGGFDPWLQDAAQALQKWREENAPWLDMPAFGLNRNHQSRLQKLARAQQEFQAQSEAYGEQLKAAIEQAFARFASRLSEHESSGSQLTSARALFDLWIEAAEESYADVALSEQFRKVYGGFANAHMRLRAALQEEVEQLSERFGMPTRSEMDAAHRRIAELERLVRRMLRNAASPASKPAAAAQPAPVTPAAGKRASSAGASAVARPAGAERAVKQGASKKAPVDKTATKKAAPKKAPAVSKPTKTTSPKKHGGATSSPELAARKPVAAKKRGAQA

SEQ ID NO: 15 - DNA, 813 bp, Xanthomonas
ttggaggcgttgttcgaccagttgcacaccttgcccaccgtgcccaaggtggcgcaagacctgatccgtcaattcgacgatccgcagaccgatatcgatacgctggcgcacagtatcgagcgcgatccggtgatcgccgccaaagtgttgcggctggccaattcggcgcgcttccatggcttgcgcgattccaccagcgtggaagacgcggccatgcggctgggcttcaacaccctgcgcacgctggtgctggcgtcggcgatgactggcgcgttccgggctggcccgggcttcgacctcaaggccttctggcggcacagtttcgaagtggccggcatctgccgcctgctggcgcgccagcagggggtggacccggagacggccttcacctgcgggatgatgcacaacatcggcgagctcttgatccagtccggtgcgccggaatacgccagccgcatcaatcacgaaacctcctctgccggtcatgcggccgaagaaaccctgcaactgggcttcggctatccggaagtcggggccgagctggcacggcgctggcagttgccggtggtgatccaggaagccatcgggtatcaggtccggcctgccgcggccccggacggtgcgcgcatgccattgctggtggcccaggcggtactggtctgcgatgcgctgcatgcgcatggcggcgccaacgcggcggcattggaagcgatacgcagcccgctgatggacggcgtggatctggacgcgttgttcgccgcgttgccggaggtcatcgaggcagatcgcgcctttgccgagttgctgcactaa

SEQ ID NO: 16 - protein, 270 aa, Xanthomonas
LEALFDQLHTLPTVPKVAQDLIRQFDDPQTDIDTLAHSIERDPVIAAKVLRLANSARFHGLRDSTSVEDAAMRLGFNTLRTLVLASAMTGAFRAGPGFDLKAFWRHSFEVAGICRLLARQQGVDPETAFTCGMMHNIGELLIQSGAPEYASRINHETSSAGHAAEETLQLGFGYPEVGAELARRWQLPVVIQEAIGYQVRPAAAPDGARMPLLVAQAVLVCDALHAHGGANAAALEAIRSPLMDGVDLDALFAALPEVIEADRAFAELLH

SEQ ID NO: 17 - DNA, 2781 bp, Xanthomonas
atgcaatttcggaccaccagccggaagacaccggttaccttgcttgcgctgtcgatcggcctggcgctgagcggccaggctgccgcacaagacgccgcccaatcgcctgccgagcccgccgtcgacctggacactgtcacggtgaccggttaccgcgccagcgtcgagaaggccctggacatcaagcgcggcgaagccggcgtggtcgatgccatcgtggccgaagacatcggcaagttccccgacctcaacctggccgagtcgctgcagcgcattcccggcgtggtgatcacccgcgacgccggcgaaggccgcgccatcacggtgcgtggcctgggcccggaattcacccgcgtccgcatcaacggcatggaagcgctcaccaccgtgggcgccggcgaccagagcggcggcaccaaccgcggccgcggcttcgacttcaacgtcttcgcctcggacctgttctcgcagctgatcgcccgcaagacggcctcggccgatgtggaagagggctcgctgggggccaccgtcgacctgcgcaccgcgcgcccgttcgactacagcggctttacgtttgccgccagtggccaggcggcctacaacgccatggccgagaaggccaatccgcgcgttgccgcgttgatcgccaatacctgggccgacaacaccttcggcgccttgctgtcggtggcctacaccgagcggcaggtgctggaagaaggctccaacaccggccgctgggccaatgggacgagcaacggcaacttcagtgcggcatcgccatttgcggccgcacgctcggcggacgtgtaccacccgcgcttcccgcgttatgtgcagatggaacacgaccagcagcgcctgggcgtgaccgggtcgttgcagtggaagcccaccgaccgcaccaccatctcgctggacgcgctgtactccaagatcgacgccacgcgcgatgagcactacatcgaggccatctccttcagccgcggtcgcgacagcggcacgccgcggcgcgccgatcgcgacggcaagccggccaccatcgtgcgcaatggcgagatccgcaacaacgcgctggtgtacggcgaattcgacaacgtcgatatccgctcggagaaccgccacgacgagtggaacaccgagttcaagcaggtcagcctggatctggagcaccgcttcagcgatgctttctccgtgtccggccgtgtcggtacctcgcgctcggcgcacgagaacccggtgcagaccaccatcatcatggacaagtacgacgtcgacgggtacagctacgactatcgcggcaacaatcgcgcgccggtgctgaactacggcatcgacccgaccaatcctgccgggtgggaactggccgaaatccgcctgcgtccgcagacggtggacaacgatttcgatacggcgcagatcgacttcaactggaacatcagccccgggttccgcctcaagggtggcgtgctggccaaggactacaccttcagcacggttgagctgcgtcgcgccagcgaaattgcggtgccgaccttcgccgatggcacccgtatcgtgccgacggacctgaccgagcaggccggcctcaagggcatcaacggcagcccgtccaactggatcgttcccaacctggatgcagtggccaaccagctcggcatctacagcaacgccggcacctacgccgtggcgccgcgcgccaacaacacccgcagcgtcgaagaaaaggaccgcggcgtctggttgatgggcgagttctccaccgacctgggctccatcccgctgtcgggtaatgtcggcgtgcgctatgtgcgcaccgagcaggagtccaccggctatgcgttgatcaacaacgcgccggcgctgaccacggtgggccgcaagtacaacaacacgctgccctcgttcaacctggtggccgagctcgcacccgacctgctgctgcgcctgggcgcggccaaggtgatgtcgcgccccggcctgggcagcctcacgcccggtgtgaccgtggcggtggccggtggcgcacgcaccgtgtccggcggcaacccggatctggatccgatcgaagccaccaatgtcgatcttggcctggagtggtacttcaacgagggcgcgatgctcggtgtcggcgtgttctacaaggacatcgaaagtttcatccagaccgcacgcgaagtgcgcccgtatgccagcagtggcttgccggcttccttgctggatggcaccggcgccagcatcaacgacgacttcaccttcagcattccgttgaatacacccggtggcgagttgaagggcgtggaagcgaactatgtccagccctttacgttcctgccgggcaagtgggccaatctgggagtgcaactgaactacacctgggtggattcgcagatccagtacctgtccagcaccggcgcaccggtgatgaagaacgacctgaccgggctgtcgcgctcgtcgtggaatgcgaccttgttctacgagggcgagtccttcgccggccgcatctcggccaccaatcgcgacgattacctgacccaggcccccggcacggaagcgggattcaacgtcgatggcgtgcacggcatgagcggcaccaccatgatcgacgcctcgctgcgctacaagatcagcaagcagctggagctgagcctggagggcatcaacctcaccaacgaagcgtccgacgaatgggtgtggtcgccgcaaaccggcgagttgccgttgcagtacaccgaaaccggccgccagtacttgctgggcctgcgctacaagttctaa

SEQ ID NO: 18 - protein, 926 aa, Xanthomonas
MQFRTTSRKTPVTLLALSIGLALSGQAAAQDAAQSPAEPAVDLDTVTVTGYRASVEKALDIKRGEAGVVDAIVAEDIGKFPDLNLAESLQRIPGVVITRDAGEGRAITVRGLGPEFTRVRINGMEALTTVGAGDQSGGTNRGRGFDFNVFASDLFSQLIARKTASADVEEGSLGATVDLRTARPFDYSGFTFAASGQAAYNAMAEKANPRVAALIANTWADNTFGALLSVAYTERQVLEEGSNTGRWANGTSNGNFSAASPFAAARSADVYHPRFPRYVQMEHDQQRLGVTGSLQWKPTDRTTISLDALYSKIDATRDEHYIEAISFSRGRDSGTPRRADRDGKPATIVRNGEIRNNALVYGEFDNVDIRSENRHDEWNTEFKQVSLDLEHRFSDAFSVSGRVGTSRSAHENPVQTTIIMDKYDVDGYSYDYRGNNRAPVLNYGIDPTNPAGWELAEIRLRPQTVDNDFDTAQIDFNWNISPGFRLKGGVLAKDYTFSTVELRRASEIAVPTFADGTRIVPTDLTEQAGLKGINGSPSNWIVPNLDAVANQLGIYSNAGTYAVAPRANNTRSVEEKDRGVWLMGEFSTDLGSIPLSGNVGVRYVRTEQESTGYALINNAPALTTVGRKYNNTLPSFNLVAELAPDLLLRLGAAKVMSRPGLGSLTPGVTVAVAGGARTVSGGNPDLDPIEATNVDLGLEWYFNEGAMLGVGVFYKDIESFIQTAREVRPYASSGLPASLLDGTGASINDDFTFSIPLNTPGGELKGVEANYVQPFTFLPGKWANLGVQLNYTWVDSQIQYLSSTGAPVMKNDLTGLSRSSWNATLFYEGESFAGRISATNRDDYLTQAPGTEAGFNVDGVHGMSGTTMIDASLRYKISKQLELSLEGINLTNEASDEWVWSPQTGELPLQYTETGRQYLLGLRYKF

SEQ ID NO: 19 - DNA, 807 bp, Xanthomonas
atgtgccgggttttgcgggtgaaccgggctggatactacgcgtggctaaagtcgcccgacagtgaacgcgccaaggaagacgaacgcctgctggggctgatcaagcaccactggttggccagcggcagtgtctatgggcatcgcaagattgccaaggatctacgcgatctgggtgagcgttgcagtcgccatcgggtgcatcgattgatgcgcaccgagggactgcgtgcccaggtgggctatggccgcaagccgcgcttccatggcggaacgccgtgcaaggcggcagccaacctgctggatcgacagttcgacgtgaccgagccggacacggcctgggcgagcgacttcaccttcatccgtacgcatgaaggctggatgtacctggctgtggtgatcgatctgttttccaggcaggtcgtcggctgggcgatgcgcgatcgagccgataccgagttggtcgtgcaggccttgctgtcggcggtgtggcggcgcaaacccagcgctggttgcctggttcattcggaccaggggtctgtctacaccagcgatgactggcgcagtttcctggcgtcccatggcttggtgtgcagcatgagtcggcgtggcaactgccacgagcgcgaagcctgcccttggggcacaacgcaccggtggagagcttctttggtctgctcaaacgcgagcggatcaggcggcggatctaccccaccaaggacgccgcacgcgccgaggtgttcgactacatcgagatgttctacaacccacaacgccgtcatggttcaactggcgacctgtcccctgtag

SEQ ID NO: 20 - protein, 268 aa, Xanthomonas
MCRVLRVNRAGYYAWLKSPDSERAKEDERLLGLIKHHWLASGSVYGHRKIAKDLRDLGERCSRHRVHRLMRTEGLRAQVGYGRKPRFHGGTPCKAAANLLDRQFDVTEPDTAWASDFTFIRTHEGWMYLAVVIDLFSRQVVGWAMRDRADTELVVQALLSAVWRRKPSAGCLVHSDQGSVYTSDDWRSFLASHGLVCSMSRRGNCHEREACPWGTTHRWRASLVCSNASGSGGGSTPPRTPHAPRCSTTSRCSTTHNAVMVQLATCPL

SEQ ID NO: 21 - DNA, 1986 bp, Xanthomonas
atggctaagcgaacaatttttcaagagcatcatattgctgaaagacaaacgttgcgcaatagccggctgcttgaggttcttcagaaagcgaattattttgaaattgatgcgcaacgcaatctgatttatatgccgagtgatccggcactagcacaggaaatcggcatatccccgcatagtggcggcccaatacgggattaccaagatggaacgatcgctcgactgaggcgtttgtcgcagaccatagacggtcaactggcgatctcgggtgatccagacgctatcgcaagaatcgcgccaaaggttgatcgactcgttgatacaatgagagtaggcctggttaatgcagatctccataccaatgccccgatcggcatggttgctgacgacatccgtcctggcatcaatgcgtttttcaagggtgcctcgagctacgatcagaagcttgcatctcagatcgacgcagttccaacgctacagagacccgaactcggctggtcgtcaatcgtccactccgagcagcgcgtcatatctacacttcagtacgtagagcaatccagcaacaacctcaccaaaggtggtgatccagaaattgggagaaattcactttcacaagccatcgcacaagctcatgcaaatggccgcctgacgctttccgaccaaggcatcctgaaggtcgaaaatgttctcggcgaagaagcagcccgaccattacgcattccgccaggtcaacgcggctttgccaccgcagagctactggccggcgatctatctgcaggccaggcactgcggactgccggcatactcgccaccgccgccgacacggtgatgaccggccagcgcgccacgcagttcctcggccaggacaacccgctcgccgcgcaatcggagcttgcgcactttgccggccgcaacgtcggtggctgggccggcggtactgccgctgcctacgcgctgggtagctcgggcgccgggccgatggtgctgatcgcagccgatgcgtatttcttgagtaccgctggcgagaaggccgccgcgttgctcgacaaccgcgccatctacacccagaccgatcgcgccggcgcgcagtggtcgttcaacggcagtgcctggacgcgcgagggcaaagccgacaccaccaacgacggggtagacaacccgacctccacccctatcgtcgccagctacgagaaggcacgcgaactcaattaccaagccaccaatgccgctgcagcgctcgccctcaaagatgcgcccgctccgcaggatccgtatcggcagccagccaatgccactgaccgtcccagcctgagcaacgccgactggcgacgcgatgcatcggacggtcaatggcatcggctggtcaaggccgagatctccggcgccaacgatcgcggcagctacatgcaggaaaccgcccagccgcaacgcgctgccgagctggaggcgcaagcgcagcacaccatcgcccgcaacatcgccaacagccccggggcgatcgctgcgcgctacgaactggcgcaccaccgcaacggctgggcggccgatggcctgccgatgtcgccagcggtgcagcaggcgctgcccgatccggatgccttgaccgcctcgaacggccagcgctactaccgcgacatcgaaggccagtggaccaacaacggcgcaccacccgacggcaaccgcgtactggaactggagaccacgcgcgcgatgctgcaaccggcactgaccgagcaggcgcaggcgattgccgccatccagcaatcgccgcaagacatacagggcgagcagacgctctaccgctatcgcattgtcggcaccgagctacagcccaatggcgtgaagcaatcgaactggcgacgcaacgcacgcgcgaggcagagggacttgcgggcgatggcgcgatgcagttgcagcgtggccctagtggacactttggcgccgatagcccgattgcgcatctgcaacgcggccccgatggcgtag

SEQ ID NO: 22 - protein, 661 aa, Xanthomonas
MAKRTIFQEHHIAERQTLRNSRLLEVLQKANYFEIDAQRNLIYMPSDPALAQEIGISPHSGGPIRDYQDGTIARLRRLSQTIDGQLAISGDPDAIARIAPKVDRLVDTMRVGLVNADLHTNAPIGMVADDIRPGINAFFKGASSYDQKLASQIDAVPTLQRPELGWSSIVHSEQRVISTLQYVEQSSNNLTKGGDPEIGRNSLSQAIAQAHANGRLTLSDQGILKVENVLGEEAARPLRIPPGQRGFATAELLAGDLSAGQALRTAGILATAADTVMTGQRATQFLGQDNPLAAQSELAHFAGRNVGGWAGGTAAAYALGSSGAGPMVLIAADAYFLSTAGEKAAALLDNRAIYTQTDRAGAQWSFNGSAWTREGKADTTNDGVDNPTSTPIVASYEKARELNYQATNAAAALALKDAPAPQDPYRQPANATDRPSLSNADWRRDASDGQWHRLVKAEISGANDRGSYMQETAQPQRAAELEAQAQHTIARNIANSPGAIAARYELAHHRNGWAADGLPMSPAVQQALPDPDALTASNGQRYYRDIEGQWTNNGAPPDGNRVLELETTRAMLQPALTEQAQAIAAIQQSPQDIQGEQTLYRYRIVGTELQPNGVKQSNWRRNARARQRDLRAMARCSCSVALVDTLAPIARLRICNAAPMA

SEQ ID NO: 23 - DNA, 993 bp, Xanthomonas
atggcccacccgcactaccgcccccgccgcatgcgccacgatgcgttttcgcgtcggttgatgcgcgaaaacacgctgaccaccgatgacctgatctggccggtgttcgtgcacgaattgtctggccgcgcgccgattgcgtcgatgccgggcgtggaacgcctgtcgctggacgagttgctgcgcgaagcggaaaacgcgctggaattgggcatcccggtgatcgacctgttcccggtgatcgacccggccggcaagagcctggatgcggccgaggcctggaacccggacggcctggcgcagcgcgcggtgcgtgccttgaagtcgcgctttccggagctgggcgtcatgaccgacgtggcgctggacccgtacaccacccacggccaggacggcatcatcgacgccaagggttatgtgctcaacgagatcacggtcgaggcactggtcaagcaatcgttgtcgcatgcgcaggcgggcgtggacatcatttcgccttcggacatgatggatggccgcatcggtgcgatccgccgcgcgctcgatgccgacgatcatctcaacgtgcgcatcatggcgtattcggccaagtacgcctcggcgttctacggcccgttccgcgatgcggtgggcagcgccggcaatctgggcaaggccgacaagaccacctaccagatggacccggccaacggcgacgaagccttgcgcgagatcgccctggatctggaagaaggcgcggacatggtgatggtcaagccaggcatgccgtatctggatgtggtgcgccgggtgaaggaggaattccgggtgccgacctttgcgtaccaggtcagcggcgagtacgcgatgctcaaggcggcagcggccaatggctggctggacgagcgcaagtgcgtgctcgaggcgttgatggccttcaagcgtgccggtgccgatggcgtgctgacctatttcgccccgcaagtggcgcgttggttgcgcgaaacacgctga

SEQ ID NO: 24 - protein, 330 aa, Xanthomonas
MAHPHYRPRRMRHDAFSRRLMRENTLTTDDLIWPVFVHELSGRAPIASMPGVERLSLDELLREAENALELGIPVIDLFPVIDPAGKSLDAAEAWNPDGLAQRAVRALKSRFPELGVMTDVALDPYTTHGQDGIIDAKGYVLNEITVEALVKQSLSHAQAGVDIISPSDMMDGRIGAIRRALDADDHLNVRIMAYSAKYASAFYGPFRDAVGSAGNLGKADKTTYQMDPANGDEALREIALDLEEGADMVMVKPGMPYLDVVRRVKEEFRVPTFAYQVSGEYAMLKAAAANGWLDERKCVLEALMAFKRAGADGVLTYFAPQVARWLRETR

SEQ ID NO: 25 - DNA, 948 bp, Xanthomonas
atgcctgattctcttcgatccattcacgtcgttgccggcgtggtcaccgacccccgcgggcgcatcctgctgacccgccgcaccgaaacccgcgacatgcctggcctgtgggaatttccgggcggcaagcgcgagcctggcgagacctccgaacaagcgctggtgcgcgagctcaacgaagagctcggcatcgaggcgcaggtgggcgattgggtgatggatgtgccacagctctatccggacaagcggctgcgcctggaggtgcgtcatatcacgagctggaaaggcagcccgcgcggccgcgaaggacaggccatgacctgggtggcggcagacaagctggcgcgttattcgatgccgccggccgacgtgccggtggtgggtgcgctacggcagcccgaccgttacctgatcacgcccgacccggaggacgacgcgcgctggctggagagcctggagcttgcattgcagaacgggatcacgcggatccagctgcgtgcgcggcagctggcgcctgcgcgttggcaggcgctgttgcagcaggtgatgcgcctgcgtggccgcgcgcgtgcgcaactgttgctcaatcgcgatatcgcactggctgcggaccttggtatcggcgtgcatctgggatcggagcagctggccggtttgcaggaacgcccgctgcccgcagagcaactcgtggccgcgtcgtgccatggcctggacgacttgcgccacgcccaacggatcggctgcgacttcgcggtgctgggcccggtgcaggcgaccgcttcgcatccgggcgccacacccatcggctgggacggtttcgagaccttgcgcgaacaggtctcgctgccgatctacgcgctcggcggcatgcaggtcgaagatgtgcgtcaggcgcgctcgcatggcgcgcagggcatcgcggcgatccgcgcgctctggccgcaatga

SEQ ID NO: 26 - protein, 315 aa, Xanthomonas
MPDSLRSIHVVAGVVTDPRGRILLTRRTETRDMPGLWEFPGGKREPGETSEQALVRELNEELGIEAQVGDWVMDVPQLYPDKRLRLEVRHITSWKGSPRGREGQAMTWVAADKLARYSMPPADVPVVGALRQPDRYLITPDPEDDARWLESLELALQNGITRIQLRARQLAPARWQALLQQVMRLRGRARAQLLLNRDIALAADLGIGVHLGSEQLAGLQERPLPAEQLVAASCHGLDDLRHAQRIGCDFAVLGPVQATASHPGATPIGWDGFETLREQVSLPIYALGGMQVEDVRQARSHGAQGIAAIRALWPQ

SEQ ID NO: 27 - DNA, 432 bp, Xanthomonas
gtgaatgcgattgccctgttcccgccgccgccgtccctggaaggacccttcgacgccaccaagtgcccagtgcgcgatgtgctcgaccagatcggcgacaagtggaccctgctgatcctgctgacgctgatccccggcccatcccgcttcagtgccatccagcgcgccgtgccggacatctccaagcgcatgctgacccagacgctgcgcaatcttgagcgcaacggcatgatcacccgcaaggtctacgccaccaagccgccgagcgtggaatacgcgctctccaagctgggaattgcgctactcggcccagtctcgcaactgctcacttgggcgggcgaaaaccacggcagcatccgcgccgcccgcgagcgattcgatcgcgcacagcaggcggcagtggcgttggatcccgacgaagccatggcatag

SEQ ID NO: 28 - protein, 143 aa, Xanthomonas
VNAIALFPPPPSLEGPFDATKCPVRDVLDQIGDKWTLLILLTLIPGPSRFSAIQRAVPDISKRMLTQTLRNLERNGMITRKVYATKPPSVEYALSKLGIALLGPVSQLLTWAGENHGSIRAARERFDRAQQAAVALDPDEAMA

SEQ ID NO: 29 - DNA, 1635 bp, Xanthomonas
ttgggacaagaccagctacgcgctgggcgtcagtgccagcagcaagcgcagcgcgcaggaacacgctgcattcaagaccttgcaccagcaggcgctcggcggcagcgaagaccccgggctgcgtgcgctattgacgttcctcgacacctggtcgcccgcgcaattcgccgatcatccacagttttcccgtcacggcgaggcgctattggataccaacctggtgttccgcctggagggcgacaccggctatctgcatcagcgcgccacggcgcgcgcggcatgggagcgactgcaagaggccaaagcgccgacggcgcatccggcatgtgcctggtcagtggcttacgcgcgccgttggcgcggctgcacccgccgatcaaaggatttgaaagcacgggatcttccatcgtttcttttaacgcggatgcttatatttcatacagaggttcgctcctaaaagttcagtcgaaacacaaggaaaatgatagcggtgccaatgcgccgatttctaaatctgcggctttcgcctacaccaccgcgctcaaccatctgctgcgccgcgacccacgcaatcgccaacgtgtgcagatcggcgacaccacggtggtgttctgggcgcaggccaggacgaccgcgcacgctgaggaggccgaagatctgatcgccgatttcctgcgcggcggtgaaacagaaggccctggcatcgccgacggccaggccacccagcgcctgcacctggcactggagcaggtgcgacaggcacgtccactacgcgaaatggacgacgcactggacgacgacgcccgcatcttcgtactgggcctggcacccaacgcctcgcgcctgtcgatccgcttctgggaaacgcagacgctggccggctttgccgcacggctggctgcccactatcaggacctcaagctcgagccgccagcctggaaacgcgcgcctaccccgcagtttctcgccctgcagacagcgccggtctacggcgagcacggcaagcccaaggccgaggacgtctcgccgcttctggcaggggagctgacccgcgccatcctcgccggcacccgctatccgcgcagcctgctcggcggcatcgtcatgcgcttccgcgccgatgggcaggtcaacccgctgcgcgtggccttgtgccgggcagtactggcacgcgaggcacgtctggacacgcaacaaggactgtcatccaccaagggagagccccccgtgagtctcgataccgccaataccgaccccggctacttgctgggccggctgttctcatcgctggaaaacctgcaacgcaccgcactgggcgggcaggtcaacgccaccatccgcgaccgttactacggcgccgcctctgcaaccccggcgagcattttcccggtgctgttgcgcaatgcgcagaatcacttcggcaagctgcgcaaagacaaggcgggcctggcggtgaacctggaaaaagaagtcggccagatcatcgacgcacttcccgccagcttcccgcgcagcctgcccatccacgagcaaggccgtttcgccatcggttactaccaccagacccaggcgcgcttcgcccgcaacaacggccaggacgcccccgacaccgcttccgaaggagaacccacatga

SEQ ID NO: 30 - protein, 544 aa, Xanthomonas
LGQDQLRAGRQCQQQAQRAGTRCIQDLAPAGARRQRRPRAACAIDVPRHLVARAIRRSSTVFPSRRGAIGYQPGVPPGGRHRLSASARHGARGMGATARGQSADGASGMCLVSGLRAPLARLHPPIKGFESTGSSIVSFNADAYISYRGSLLKVQSKHKENDSGANAPISKSAAFAYTTALNHLLRRDPRNRQRVQIGDTTVVFWAQARTTAHAEEAEDLIADFLRGGETEGPGIADGQATQRLHLALEQVRQARPLREMDDALDDDARIFVLGLAPNASRLSIRFWETQTLAGFAARLAAHYQDLKLEPPAWKRAPTPQFLALQTAPVYGEHGKPKAEDVSPLLAGELTRAILAGTRYPRSLLGGIVMRFRADGQVNPLRVALCRAVLAREARLDTQQGLSSTKGEPPVSLDTANTDPGYLLGRLFSSLENLQRTALGGQVNATIRDRYYGAASATPASIFPVLLRNAQNHFGKLRKDKAGLAVNLEKEVGQIIDALPASFPRSLPIHEQGRFAIGYYHQTQARFARNNGQDAPDTASEGEPT

SEQ ID NO: 31 - DNA, 1737 bp, Xanthomonas
atgcgtagcactcccaatcgcgcagcgcccggcctgatcctggccgccaggcaattgcataacgacagactggtgcggctgttcgagtcgttcctggaatatcgcgcctgcgtcggccgtgtgatcgtgcagaccatcgtcactgcgtggtgcctgggctggctgtacgggccgcaaccggtgctggtggccgacgcgcaccatgtctttcccactgcgctgagcctgtgggcgctgtcggtggtgtggatgctgctggtgcgccgccgtgtcattgcgcccagcgaatggctcgatgcgatggggtttgcgatgaacctgctgttcatcggcatccagacctacctggccttcatcctgctgatgtcgttgaacgcatttttgccgttcatcaccatcgctgcggtggcgcgttacgggcaacgcgcgatatttccggtgttgctcagcaccttcgtgctgatgctgttgaccgcaccgtcaggctactggctgtcgcgtccggcatatttcgtctatgccgtggcgctgatcatcgtgctgccgttgctggtggcgcggatcgtgcttgcgatgcaggaagtggcactgcaggcgctggcctcgcgcgatgcgcaaagccatttcatcagcaccatgagccacgaattgcgcaccccgctgaatgcggtgatcaactgcgcccagttgatcgagaccgaacaaatgccggcgcagcaacgcgagttgctgcaatcggtgaccgccaatgcgaccgcactgcgccatcgtgtcaacgaggtgctggacgtggccagcatcgatggcgggcgcctgcagctgcatcgcaagcccttgaacctgctggacgtggtcaccacggtgaaagcggtctgcgccaccgcggcgtccagcaagggcgtcacgctcgatgtgcgtgtcgatagcccgcaagcaccgcatgtgatgggcgacgaaggccgcatcgaacaggtgatcagcaacctggtgatcaacgcgatcaagttcaccccggccggcggcacggtggatctgctgatcgacgcgacccaggtgcaggagcgctggagcatcgcagtgaccgtcaccgacaccggtatcggcgtaccggacgatcagaaggcctacatcttcaccccgttcactcagctcagtaccggcttcagtcgcgtcgagggcggcgtgggcctgggcctgtacatcgcgctgtccgtctccgatgcgatgggcggcagcctcaccgtgagcgacaaccccgccggtggcagcatctttcgctggatcttcgaactgcccggcaccgcggccggtagcaatacgctcgcactgcgcgatgcgctggcccagcacgcacaaaacgtaccctccctgcattgcctggtctttgaagacatggataccaatcgtctggtgatcggcaatctgctgacccgcgccgggcatcgggtgagcttccaggtcgatggcaccgatgcggtgcagcgcatccgcgaggcagcgccggacctggtgttcctggacctgcacatgcccggtacgtccggctgggatgcgctgcgcgaagcgcgcgatgcgatgtcggcgctgccgccgatcatcgtgctgaccgccgacacccggaccgattcgatgcgcgacgccagcgcggcgggcgtggccggctacctgcccaagccgatcaatgcgcacgagttgctggcattgctcgcccagcacgccagccatgcacgcccctga

SEQ ID NO: 32 - protein, 578 aa, Xanthomonas
MRSTPNRAAPGLILAARQLHNDRLVRLFESFLEYRACVGRVIVQTIVTAWCLGWLYGPQPVLVADAHHVFPTALSLWALSVVWMLLVRRRVIAPSEWLDAMGFAMNLLFIGIQTYLAFILLMSLNAFLPFITIAAVARYGQRAIFPVLLSTFVLMLLTAPSGYWLSRPAYFVYAVALIIVLPLLVARIVLAMQEVALQALASRDAQSHFISTMSHELRTPLNAVINCAQLIETEQMPAQQRELLQSVTANATALRHRVNEVLDVASIDGGRLQLHRKPLNLLDVVTTVKAVCATAASSKGVTLDVRVDSPQAPHVMGDEGRIEQVISNLVINAIKFTPAGGTVDLLIDATQVQERWSIAVTVTDTGIGVPDDQKAYIFTPFTQLSTGFSRVEGGVGLGLYIALSVSDAMGGSLTVSDNPAGGSIFRWIFELPGTAAGSNTLALRDALAQHAQNVPSLHCLVFEDMDTNRLVIGNLLTRAGHRVSFQVDGTDAVQRIREAAPDLVFLDLHMPGTSGWDALREARDAMSALPPIIVLTADTRTDSMRDASAAGVAGYLPKPINAHELLALLAQHASHARP

SEQ ID NO: 33 - DNA, 1437 bp, Xanthomonas
atgcgaccgctgcctaccctgctgactctttcgctggccgctgccttcggcggctttgccgccaccggcatcaacgcctggctcgacaatcgcgccgaagccacaccccccagcaacgccgcctttaccctgccgacggccgccgcattgccggcggcggtggccggccaacctgtgccgtcgctggcgccgatgctgcagcaggccatgcccgcggtggtgagcgtgaacaccaaacaggtggtgcgcgtccgcaacccgttcttcaacgacccgatcctgcgccggctgtttccgcaggtgccgcaggaccgcatcaacgagtcgctgggttcgggcgtgatcatcgatgcgcaaaaagggtatgtgctgaccaatcaccacgtcatcgaaaacgccgacgacgtgcaggtgacgctcggcgacggacgcacggtcaaggccgagttcatcggctccgatgccgataccgatattgcgctgatccgcatcaaggccgacaacctcaccgatatcaaactggccgacagcaacgcgctacgcgtgggcgattttgtggtggccatcggcaacccgttcggcttcacccagaccgtcacctccggcatcgtctcggcagtggggcgcagcgggattcgcggcttgggctaccagaacttcatccagaccgatgcctcgatcaaccccggcaactcgggcggcgcgctggtcaacctgcaaggccagttggttggcatcaacaccgccagcttcaatccgcagggcagcatggccggcaatatcggcctggggctggcgattccgtccaacctggcgcgcaatgtcgtcgagcaattggtgaccaagggcgtggtggtgcgcggcacgctcggcctggagacccagaacctcacccaacagatgctgcagggcctgggggtggattcgttgcgcggcgcattggtgacacgggtgttgccgggctccgcagcggcggcggcaggcgtgcaaccgggcgatgtcgtggtggcggccaacgaccagcgcgtggacagcgccgaagcgctgcacaactacgaaggcctgcaggcggttggcagtgcggtgaccctggatatccgtcgcgatggcaagccgctcaaactcaaggccacgctgagagagcaggaccgcgccatcaccggcgacatgctggacccgcgtctgaccggcgcgaccttcgtcgatctccccgaatcgctgcgtcagtcgggcatcaccggggtgatggtgagcgaggtcaaacgcggcggtcgcgctgccgccaacggcctggtcgccggcgatgtgatcgtggccagcagcgtgggcgagttcgccgaccttgccagctggcgggccaatttccaacgtaaaccgcagcagcttgtcgtgcgtatcctgcgtggcaacgcgcagtacgacgcgctgatgcgttga

SEQ ID NO: 34 - protein, 478 aa, Xanthomonas
MRPLPTLLTLSLAAAFGGFAATGINAWLDNRAEATPPSNAAFTLPTAAALPAAVAGQPVPSLAPMLQQAMPAVVSVNTKQVVRVRNPFFNDPILRRLFPQVPQDRINESLGSGVIIDAQKGYVLTNHHVIENADDVQVTLGDGRTVKAEFIGSDADTDIALIRIKADNLTDIKLADSNALRVGDFVVAIGNPFGFTQTVTSGIVSAVGRSGIRGLGYQNFIQTDASINPGNSGGALVNLQGQLVGINTASFNPQGSMAGNIGLGLAIPSNLARNVVEQLVTKGVVVRGTLGLETQNLTQQMLQGLGVDSLRGALVTRVLPGSAAAAAGVQPGDVVVAANDQRVDSAEALHNYEGLQAVGSAVTLDIRRDGKPLKLKATLREQDRAITGDMLDPRLTGATFVDLPESLRQSGITGVMVSEVKRGGRAAANGLVAGDVIVASSVGEFADLASWRANFQRKPQQLVVRILRGNAQYDALMR

SEQ ID NO: 35 - DNA, 630 bp, Xanthomonas
atggacaacacgcggatcgacaatgcggcacgatgtgttcagcgctggacggacgcgttggcgcacctggccgaatcgttggacgccgcacgggtggcgcattggcttggcccggtggcggcgtcgggctgggcagcggcgcccgtctaccgtcgtatgcgggtgatccaggcgtggtcgggatggtcgggattgtcgccctctgcgctcggcctgcatgcgaaccaactcgccgtcctgatgcccgcgctattggccaaggtcctgatcgcgcgtgcgctgttctcgcgcgggttggcagtgcgtcgttgtatcgagcgcgagcgtctggactggctcgaacagtgcgtgggtcctgccgtgcttgagcacgtgcgccagaacgcgtccgccggcctacctgtgccgctgttaccgcgcgatgccgatcaagcggcgtgggtcggcgacggttggcgtcggatgcaggcagatggcgtctggcccaacccggttgttgccaagctgctggcgctgtccttgcccctgggcgccgcgcaggtggcggctctcgcagcagacggtgcgagcgatgcattcctggccgcgcttcctaccctgatgccggaggcgtcatgcgtctctggttga

SEQ ID NO: 36 - protein, 209 aa, Xanthomonas
MDNTRIDNAARCVQRWTDALAHLAESLDAARVAHWLGPVAASGWAAAPVYRRMRVIQAWSGWSGLSPSALGLHANQLAVLMPALLAKVLIARALFSRGLAVRRCIERERLDWLEQCVGPAVLEHVRQNASAGLPVPLLPRDADQAAWVGDGWRRMQADGVWPNPVVAKLLALSLPLGAAQVAALAADGASDAFLAALPTLMPEASCVSG

SEQ ID NO: 37 - DNA, 1185 bp, Xanthomonas
atgcgaacgcaggttgcgatcatcggcgccggaccatcgggtttgttgctgggcgaactgttgcggctcgccggcatcgactgcgtcatcatcgagcggcagacgccccagcatgtgctggcgcgcatccgcgccggggtattggagcagggcagtgtggaactgctgcagcgggccggcgtcggcgagcggttgcagcgcgagggcttgttgcatcacggcttcgaattgtgcctcgacggtcagcgcgagcgcatcgatctgctgcgcggctgcggacgtggcgttacggtctacgggcagaccgaggtcaccgccgatctgatgcacgcgcgccaacgcagcggggcgcccacgcattacaacgcgcaggatgtgaccctgtgcgatgtggaaggcgcgacgcccgccgtggagttcacacaggacggccagcgcaaacgcctccactgcgactacatcgtcggctgcgatggttatcacggcatcagccgtgccagcatccccgccgagcggctgcgtctgttcgagcgcgtgtacccattcggttggctcggtgtgctggccgatacgccacccgtgcacgaggaactgatctacgcacgccacccgcgtgggtttgcactatgttcgatgcgctcgcccacccgtacgcgctattacatccaggtgccagccagcgcacaggtggaggcctggagcgatcaggcgttctgggacgagttgcgtgcgcggctcccgcctgcgttggcggcgcaactggtcaccggtgcatcgatcgaaaagagcatcgcaccgctgcgcagtttcgttgccgagccgatgcagtatggtcggctgttcctggccggcgatgctgcgcatatcgtgccgcccaccggcgccaaggggctgaatctggcgcttggcgatgtcggcttgttggcgcagctgttcgagcgttggaaggccagcggcgatgctggtgcgctggagcagtattcgacattggccttgcagcgggtgtgggaggccgaacgtttctcgtggtggatgaccacgctgttgcaccgtttcgacgacgaagacgcattcaccgcgcggctacacggagcggagctggactatctgctcggtagcgaggcgggccgcgcgacgctggcggaaaattatgcaggtctccccttggctgcgctgtctgattga

SEQ ID NO: 38 - protein, 394 aa, Xanthomonas
MRTQVAIIGAGPSGLLLGELLRLAGIDCVIIERQTPQHVLARIRAGVLEQGSVELLQRAGVGERLQREGLLHHGFELCLDGQRERIDLLRGCGRGVTVYGQTEVTADLMHARQRSGAPTHYNAQDVTLCDVEGATPAVEFTQDGQRKRLHCDYIVGCDGYHGISRASIPAERLRLFERVYPFGWLGVLADTPPVHEELIYARHPRGFALCSMRSPTRTRYYIQVPASAQVEAWSDQAFWDELRARLPPALAAQLVTGASIEKSIAPLRSFVAEPMQYGRLFLAGDAAHIVPPTGAKGLNLALGDVGLLAQLFERWKASGDAGALEQYSTLALQRVWEAERFSWWMTTLLHRFDDEDAFTARLHGAELDYLLGSEAGRATLAENYAGLPLAALSD

SEQ ID NO: 39 - DNA, 1134 bp, Xanthomonas
atggatatcggctacttcctgaagctgatgaccgaaaagaatgcctcggacatgttcttgaccaccggcgcgccggtgtacatcaagatcgaaggcaagctttacccgctcggcaatacggggttgccgccggggatggtcaagaaaatcgcttactcgttgatggacgaaggccaggtaccgcagttcgagcgcgagctcgagctcaacatggccatcgccctgccggacgccggccgcttccgcgtgaacgtattcaagcagcgcggcgaggtgggcatggtgattcgcgccattcgcagccggattcccagcatcgaagagctcaacctgccgcaggtgctcaaggacgtcatcatgaccccgcgcgggctggtgctggtggtgggttcgaccggctcgggcaagtcgacctcgctggcgtcgatgatcgaccaccgcaacagcaccaccaccgggcacatcctcaccatcgaggacccgatcgaatatctgcacaaacacaagatgtcgatcgtgaaccagcgcgaggtgggcctggacacccacgccttccagagcgcgctgaagaacgccatgcgcgaggcgccggacgtgattctgatcggcgagatcctggacgccgaaaccatggaggcggcgatcgccttcgccgaaaccggccacctgtgcctggccacgctgcactccaacaatgccgaccagaccatcgagcgcatcctcaatttcttcccggaaagcgcgcacaagaacgtgttgatgaacctggcgctgaacctgcgcgcggtcatttcccagcgtttggtcaagggcgtggacggccgtcgtcgaccggccaccgaagtgctgctcaacacgccgatgatccgcgacctgctgcgccgcggccaagtgcacgaggtcaagcaggcgatggaagagtcgctggaagaaggcatgcagaccttcgaccaatgcctgttccggatggtgaagtcgggcgaaatcgagcaggaagaagcgctgcgcgctgccgactcgcgcgacggcctggccctgaagttccgtctttccgaaggcggctccggcgagcacgacccgtacgcggactacgaagcggccgcggcaccgaagatcagccacgggttcatctaa

SEQ ID NO: 40 - protein, 377 aa, Xanthomonas
MDIGYFLKLMTEKNASDMFLTTGAPVYIKIEGKLYPLGNTGLPPGMVKKIAYSLMDEGQVPQFERELELNMAIALPDAGRFRVNVFKQRGEVGMVIRAIRSRIPSIEELNLPQVLKDVIMTPRGLVLVVGSTGSGKSTSLASMIDHRNSTTTGHILTIEDPIEYLHKHKMSIVNQREVGLDTHAFQSALKNAMREAPDVILIGEILDAETMEAAIAFAETGHLCLATLHSNNADQTIERILNFFPESAHKNVLMNLALNLRAVISQRLVKGVDGRRRPATEVLLNTPMIRDLLRRGQVHEVKQAMEESLEEGMQTFDQCLFRMVKSGEIEQEEALRAADSRDGLALKFRLSEGGSGEHDPYADYEAAAAPKISHGFI

SEQ ID NO: 41 - DNA, 2031 bp, Xanthomonas
gtgcagggtctggacatcaccgaccgcatcagcctggaacgcgcggtgcgcgaggccgaagtgcgcaaccggcagatcctggacagcgtgatggactacgccatcatcgccaccgatatgcacgggcgggtgacgtcgtggaacgagggtgcccggcgcattctgggctggagcgaggccgagatgctcgggcaaacgctgcagcgcacctttactccggaagacgtcgaacagcggcaactcttgatcgaagcggcggcagcgctggaaagcggcagcggcatggacgagcgctggcacgtgcgcaaatccggcgagcggttctgggccaacggctcgctgatggtgttgcgcgaagaaaccggcgaggcgatcgggttcgtcaaggtgttgcgcgatcgcaccgccgagcggctggccagcgaggcgctgcaaaagagcgagcggcgcctggacgcactggtgcgcgcgtcgtcgcagtcgattttttccgccagtgcccactggcgcgaattgcgccaactggtgggcgacggcgtgctgagcgacgcggtgtcggccacgcccgactggcagacgatggtgcacccggacgaccgcgcgcggttggcgcaagcgcttgcgcaggcgactgccaccaagtctggtgtggatgtggaataccgggtgctgggcgcagatgggcatgtgggttggaccttgatgcgcgcgattccgttgctcgatgagcgcgagcagatcgccgaatggttcggcacctctgccgacatcaccgacaaacgcgtggccgaagagcagctgcgtcaacttaccgagacgctggaagagcgcgtgcgcgaacgcagcgcggcgttgttgcttgccgaagagaagctgcgtcaaagccagaagatggaggcggtggggcagctgaccggcgggctggcgcacgacttcaacaatctgctcaccgccatcagtgttggcctggaactgctgcagacgcgcatcgagcagggcaaatacgaccggctggagcgctatgtggaaatggcacagtccagcgcggcgcgcgccaccgcactgacccagcggctgctggcgttttcgcgccggcagacgctggcgcccaccgcactggatgtggaagcgctggtgcgtggcatgcacgacatcattgcgcgcacgctggggccgtcgatcgcgctgcagctgcgtccggcggaccaagcatggcaggtgctggtggacgcgccgcagctggaaaatgcgctgctcaatctgtgtatcaacgcgcgcgacgccatgcccgatggtgggcagctcaccatcagtctgcacaaccgcgcgctggatgccgctgccgcgcagcagctggagctgccacctggcgattacgtgtgcctgagcgtgcaggacaccgggaccggcatgacggccgaggtcatggccagggtgttcgaaccgtttttcaccaccaagcccatcggccagggcaccgggttgggcctgtcgatgatctatgggttcacccgccagtccggcgggcatgtgcgcatcgattcggaagtgggcgtaggcaccacaatggccttgtacctgccgcgtttcaccggcgtgctggcgcaggacgacatggcacctgccgatgcgccgctgcagcgcaacgcggcgcacagtcgcaccgtgttgttggtggaggacgaaaccgcgattcgtgtgttgatttccgaagtactcaccgaagccggataccgcgtgatcgaaaccgccgagggcagtgcggcggtggagcgcttgcgctcgcaagagcacatcgatgtgctggtgaccgacgtcggcctgaccggcggcctcaacgggcgtcaggttgccgatgccgggcggcaatcgcgtcctgcattgccggtgctgttcgtcaccggctatgcggccagtgccgcagtgggcgcgggccagctcgacgatggcatggaggtgttgaccaagccattccaggccatcgagctggagcggcgggtggcgcagttgctggagcgtcgcgcccactaa

SEQ ID NO: 42 - protein, 676 aa, Xanthomonas
VQGLDITDRISLERAVREAEVRNRQILDSVMDYAIIATDMHGRVTSWNEGARRILGWSEAEMLGQTLQRTFTPEDVEQRQLLIEAAAALESGSGMDERWHVRKSGERFWANGSLMVLREETGEAIGFVKVLRDRTAERLASEALQKSERRLDALVRASSQSIFSASAHWRELRQLVGDGVLSDAVSATPDWQTMVHPDDRARLAQALAQATATKSGVDVEYRVLGADGHVGWTLMRAIPLLDEREQIAEWFGTSADITDKRVAEEQLRQLTETLEERVRERSAALLLAEEKLRQSQKMEAVGQLTGGLAHDFNNLLTAISVGLELLQTRIEQGKYDRLERYVEMAQSSAARATALTQRLLAFSRRQTLAPTALDVEALVRGMHDIIARTLGPSIALQLRPADQAWQVLVDAPQLENALLNLCINARDAMPDGGQLTISLHNRALDAAAAQQLELPPGDYVCLSVQDTGTGMTAEVMARVFEPFFTTKPIGQGTGLGLSMIYGFTRQSGGHVRIDSEVGVGTTMALYLPRFTGVLAQDDMAPADAPLQRNAAHSRTVLLVEDETAIRVLISEVLTEAGYRVIETAEGSAAVERLRSQEHIDVLVTDVGLTGGLNGRQVADAGRQSRPALPVLFVTGYAASAAVGAGQLDDGMEVLTKPFQAIELERRVAQLLERRAH

SEQ ID NO: 43 - DNA, 1794 bp, Xanthomonas
atgactgaaacttttcttgtgtacgcgctggccatcgcgctggcgtggcccctgggccgttacatggccgcggtgatgcgcggcgcaccgatgcgtggcgatgcgctgttcggctggatcgaacggccgttgtacgcactgctcggcacgcgcccgcagcagggcatgtcgtggcgcggctacagcatggcttttctgctcagcaaccttgtcgttggtttgttgacctgggtggtgttcatgacccaggcatggctgccgttcaacccggatgccatccccaacatgcgttgggataccgcgctgcacaccatggtgtcgttcgtcaccaataccaatcagcagcattattccggccaggcgcagttgtcgtatctggcgcagatgaccggcatcgtcggcctgcaggtggtgacgccgatgatgggattggcattggcggttgcgacgctgcgcgccttgttcggtgggcgcgcagccacacaagcgtcgattgccgcaccgattctggacacgacggctgtggcggagatttcggagccggaagtgtcgcgtcagggacgccaacacacggggtccggcctggaagcgctgcctgaagccgacgtcggcaactattgggccgacgtgatccgctccagcgtacgcgtgttgttgccgctgtgcctgctgtggaccgtgctgctcggctggcagggcgtgccgtcgaccttgcagggcgcggccaccgcgacgccgttggatagcagcgcgggcatgcccaagcagaccattgcggtgggcccggttgcggcgatggtggcggtgaaacagctcggcaccaacggcggtggctggtatggccccaacagttcagtggcgctggaaaaccccacaccgttgagcaatctgctcgaaaccctcgccttggtcttgctgccgatgagcgtggtgttcatggtcggctatctgaccgggcgcaagcgacttacggcgttggtgttcggcacgatgttgacgatgtccgccgcatccaccgcgctcttgctttggtccgaagcgcactcggccaccgcaagctcgccggcgctgatggaaggcaaggaagtgcgcatcggtgcggacgcctcggcattgtgggcggcgttgaccacgcagacctccaatggctcggtcaatgcgatgcacgattcgttggcgccgttgagtggcctggtcacgttggtcgacatgctgatcaacgccatctggggcggcatcggctgcggcctgcagcagttcgtggtgtatctgttgctgagcgtgtttctggccggcttgatgactggacgcacgccggagttgttcggccgcaagatcgaatcgcgcgaagtgcagttgctggcgctgttgatcctgctgcagccgctggtggtgctgggctttaccgcagtggcgctggcagtcccggcattgactgccaattccaaccccggcttccacggcatcagccaggtgttctacgagtacacctctgcgttcgccaacaatggctcgggtttcgaaggcctgggcgatgccacgtactggtggaatctgagctgctcagtggtgctggcgctgggacggtatccggcgttgatcctgccgttgatggtggccgcacgcatggcagtgaagcggcgtgcaccggagtcggccggcagcctgcaggtcgaaacgcccacctttgcgctgaccctgatggcgatcgtgatcgtgctgaccgtgctgcagttcatgccggcactggtgctgggtcctatcgctgaacacctcgccctggcggcgtcctga

SEQ ID NO: 44 - protein, 597 aa, Xanthomonas
MTETFLVYALAIALAWPLGRYMAAVMRGAPMRGDALFGWIERPLYALLGTRPQQGMSWRGYSMAFLLSNLVVGLLTWVVFMTQAWLPFNPDAIPNMRWDTALHTMVSFVTNTNQQHYSGQAQLSYLAQMTGIVGLQVVTPMMGLALAVATLRALFGGRAATQASIAAPILDTTAVAEISEPEVSRQGRQHTGSGLEALPEADVGNYWADVIRSSVRVLLPLCLLWTVLLGWQGVPSTLQGAATATPLDSSAGMPKQTIAVGPVAAMVAVKQLGTNGGGWYGPNSSVALENPTPLSNLLETLALVLLPMSVVFMVGYLTGRKRLTALVFGTMLTMSAASTALLLWSEAHSATASSPALMEGKEVRIGADASALWAALTTQTSNGSVNAMHDSLAPLSGLVTLVDMLINAIWGGIGCGLQQFVVYLLLSVFLAGLMTGRTPELFGRKIESREVQLLALLILLQPLVVLGFTAVALAVPALTANSNPGFHGISQVFYEYTSAFANNGSGFEGLGDATYWWNLSCSVVLALGRYPALILPLMVAARMAVKRRAPESAGSLQVETPTFALTLMAIVIVLTVLQFMPALVLGPIAEHLALAAS

SEQ ID NO: 45 - DNA, 429 bp, Xanthomonas
ttggacttcctggaggacatcaaggcttgcgggcaccctcgcctcttcccccacctctctgcaggcgtcaaccgggagaccggtgagacgaatgcgcgctacagccaaggcgcggtgaaccagttcagcagctacatgaagaccttgggcttcggaaagggcatcggcgctcatgcgttccgccacacgttggccactgagctgcaccacaagaacgtatccgaccaagacatcgccctcatcaccggccactcgctgcggaaaaacgtgcccgtcttgcatgacgcctatttccacaagaagcccaagctcgcccgggccaagcaaatcaaaattctggcgaaatacaagccgccggtcgagttgcccaagtatgagcgtgggcagtttaaggagagcttggcagacccaagtaagttctatccgtaa

SEQ ID NO: 46 - protein, 142 aa, Xanthomonas
LDFLEDIKACGHPRLFPHLSAGVNRETGETNARYSQGAVNQFSSYMKTLGFGKGIGAHAFRHTLATELHHKNVSDQDIALITGHSLRKNVPVLHDAYFHKKPKLARAKQIKILAKYKPPVELPKYERGQFKESLADPSKFYP

SEQ ID NO: 47 - DNA, 360 bp, Xanthomonas
atggggcgtcacaacgctctaccttgggtggtattgaacgctccacactcgctctggcgggctagttccccgctaggggcctatgcccaccacgcaggaaacggcggcatcgccagggcaatcggcgctgggggcgtcaagacctattcgtgtcggtgctccagcctgcgacaccgacccaccgacgtccagaacttcgaactgatcggctgcaccaaggccgaggacgccgacggctaccgttgcgatacccgtggccaggtgaccctggaaatcgccggcaggcaggttcccgtccccgtcagcaaaaatctccgctatgcgaaggccgacggcacgtggagggcctacgccaaatga

SEQ ID NO: 48 - protein, 119 aa, Xanthomonas
MGRHNALPWVVLNAPHSLWRASSPLGAYAHHAGNGGIARAIGAGGVKTYSCRCSSLRHRPTDVQNFELIGCTKAEDADGYRCDTRGQVTLEIAGRQVPVPVSKNLRYAKADGTWRAYAK

SEQ ID NO: 49 - DNA, 447 bp, Xanthomonas
atgcctaaatctgacctgacacaggccttgacggctatagagccgaaaaccaaggcggctaaggtgcgcgaagtgatgccggtgattgaacaaaggatcgcggcaggtgtgcgtatcgcggacattctcaagaccctgaaagacagcgggattgatctgaccgaagccacactaaagagctatctgtatcgataccgaaagaagcatcaagcaaagaccaaagggcagcaatcggcctcgcagacggtcggcatgccggcaccacctcaacctggcgaatccgaatctgtatcgcacgaaacagattcagccgaaacaccaccatctcgcggtccgatctcgatgcaagagcttgaccgcgtgatgaaacccgacccggaagaacaggccaaggatatcgcccgttacgagcgcttggccaaagagaaatggagaaagcacaaatga

SEQ ID NO: 50 - protein, 148 aa, Xanthomonas
MPKSDLTQALTAIEPKTKAAKVREVMPVIEQRIAAGVRIADILKTLKDSGIDLTEATLKSYLYRYRKKHQAKTKGQQSASQTVGMPAPPQPGESESVSHETDSAETPPSRGPISMQELDRVMKPDPEEQAKDIARYERLAKEKWRKHK

SEQ ID NO: 51 - DNA, 1734 bp, Xanthomonas
atgggcaaccgcaagcacgtggctgcgccattcaacgacgaccaggtcatggatgtgctgttggcgcgtgggcagccggcccggggcggtgtggccggcgtctctctcctgtcgccattccaggcgttccgcagcctgtggtggcaacactgggacccgcgcccgaaggatcagcgtcggcagcactggttggcgatgcttggcagctttctcatccacctcgggtttatcgcgctgctcgtctgggtggtcaccgtgcgctgggcgccggaggaaaccacgcccggcgacgattcgcgagtccgcatgaccttgatcggcgatggggcggaggagcagggcggcggggaagggcaacctgcggctgaggcatcgacgcaggcggctgcagctgcggcgtcttcgccagcgcacgcgggtgcatcggcggcgaaaagcgagccagctccattgtcgacaccgacaccgacaccagcgcagccagcggccgatcctgtcgagcgccccgacacggcccaggcaccggaacatgcgccggaaccggtgcaagctgccagcgaaaccgtcgccgcggatattccgcaggtgacggtacaggtgccgtcggtgacgatcgaatcgccgctgcaggtcaccgaaacccaggtcgccaccaccgactttgtcgtaccaccgccgccaacgatcacgctgacgccgcgtgcgatcgaacgcgcagcaccgcaggtcgaggtgcgtcaacgcgacatccagacggtgaccgaacgtccgcaggtgcgcgaactgcaacgtccggccacggatgtagcgatgcgcaccgccacggccccaacggtgcgcgagcgcgacatcgtcatccccgagcgcccgcaaatcgccgcgccggctgcacgcctgcgtgagatctcgccgacggtgcgcatgcctgacgtcgcggtgcgtcctgcagagctgccgaacgtaccggatccgacgcctgcgccagtggcgacacctgtcgtgccagcggcgcctgcgcctgcgcctgcgactcccgacccgcagccggccaccgtggcggcgccatccgcgcaagcagccgcgcaacctgcgcagtcgcgggccagtcctgctcagtccgagcgttccagcagcgcggcggccgctgcatcgtcgcccgtcaagcccgccgccagcagtcatgccggcccgacgcctgccgatcgcagcggcgggtgggacgtggccgccaacgccgacgactggagcaaatccgaccgcaatcggcaaggcgacacgacaggtgccaacacccagcgcaacggcatgttcaatgccgatggcagcgtcaaggtggccaccggaaccggtgatgccggcaaggcgacaggtgaccgcggaccaccaggcagtgagaccgatacctggacccgcgaccagattgcgcagggcggcacctggcttaaacgtccgccgtacggctacacgccgacatcgctggacaagtactggatgccgaaccagacgctgctgcaggagtgggtgcggcgcggcctgaagaagatcgagattccgattccgggcacgaccaccaagatcagttgcgtggtgtcgctcctgcaattcggcggcggttgcggcctgagcgaccccaacctcaacgatcaaccggcaaccgcacgcccaccaccggacgtgccgttcaagcgggagctgcaggaagacaagggcgcgctgccccataacggcgggtga

SEQ ID NO: 52 - protein, 577 aa, Xanthomonas
MGNRKHVAAPFNDDQVMDVLLARGQPARGGVAGVSLLSPFQAFRSLWWQHWDPRPKDQRRQHWLAMLGSFLIHLGFIALLVWVVTVRWAPEETTPGDDSRVRMTLIGDGAEEQGGGEGQPAAEASTQAAAAAASSPAHAGASAAKSEPAPLSTPTPTPAQPAADPVERPDTAQAPEHAPEPVQAASETVAADIPQVTVQVPSVTIESPLQVTETQVATTDFVVPPPPTITLTPRAIERAAPQVEVRQRDIQTVTERPQVRELQRPATDVAMRTATAPTVRERDIVIPERPQIAAPAARLREISPTVRMPDVAVRPAELPNVPDPTPAPVATPVVPAAPAPAPATPDPQPATVAAPSAQAAAQPAQSRASPAQSERSSSAAAAASSPVKPAASSHAGPTPADRSGGWDVAANADDWSKSDRNRQGDTTGANTQRNGMFNADGSVKVATGTGDAGKATGDRGPPGSETDTWTRDQIAQGGTWLKRPPYGYTPTSLDKYWMPNQTLLQEWVRRGLKKIEIPIPGTTTKISCVVSLLQFGGGCGLSDPNLNDQPATARPPPDVPFKRELQEDKGALPHNGG

SEQ ID NO: 53 - DNA, 1547 bp, Xanthomonas
taagtgaagagtttgatcctggctcagagtgaacgctggcggcaggcctaacacatgcaagtcgaacggcagcacagtaagagcttgctcttatgggtggcgagtggcggacgggtgaggaatacatcggaatctactctttcgtgggggataacgtagggaaacttacgctaataccgcatacgacctacgggtgaaagcggaggaccttcgggcttcgcgcggttgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccatgccgcgtgggtgaagaaggccttcgggttgtaaagcccttttgttgggaaagaaaagcagtcggttaatacccgattgttctgacggtacccaaagaataagcaccggctaacttcgtgccagcagccgcggtaatacgaagggtgcaagcgttactcggaattactgggcgtaaagcgtgcgtaggtggtggtttaagtctgttgtgaaagccctgggctcaacctgggaattgcagtggatactgggtcactagagtgtggtagagggtagcggaattcccggtgtagcagtgaaatgcgtagagatcgggaggaacatccgtggcgaaggcggctacctggaccaacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggtcttgacatccacggaactttccagagatggattggtgccttcgggaaccgtgagacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggtagggacagagggctgcaaacccgcgagggcaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctggatcacctccttt

SEQ ID NO: 54 - DNA, 4533 bp, Xanthomonas
ttggacgcgatgcaggacgtgcagaagcgagccccgggatacgggagcaagtggttcgggatgttgttggccattgtggtgacgtgcgtttccctcgttcctacgatacagatcgcccacgcctatgactcgggcgtctgcaatgcagggacgcgcttgtgcgatcaaggccaggcttatctcaatgccatcaaggaaggaaacgaagcaatctgcattggcggcgggataaatacacacgccagcttggtactcgacctagcctatgatcagcgctaccaagcggtagttcagtgctacagagcgtttgataactttaccagcgaatctggtttaggcgaaagcttttatcccctaaagtgcagcgccagaccgacattcacccctgctgctggcagtactggcaacctatgccaggaaggctgcgaatatacggtcagcaacaacggcggccagccggtcgcaacgccaaacggggcggtctgctcgccaccgccaccgctcgatcccggcaagaacaatggctgttgcgatgggagcactggtccgaccaccgccggcaatccggccaatgtgttcaccggaaccaagctcgaaacggcggccgactaccgtgcgccagcagggcatctggaattcgtccggtattacagcagtgccgtcggcctgcagcccaccagcgcgctcggcaacacctggcgccatagctacatccggaccatcaccgtcaccgatgcatcgacactgatcctgacgcgccccaacggtaacttctacacgttcaagaagtccgccaatggcacatggacaccggactgggatgtgcgcgaaacgctgtccgaagtgcaagacaacgggacgctgactggatggcaggtgactgccaccgacgacagcaaagagcagttcgatctggacggcaagctgaccggcatcagctacaccgatggcgagcagctcaccctgacttacgccggccgccagttgcaatcggtgacggatacgcgtggccgtcgcctgctatttgcctaccaggcagacaggatcacacaagtcggactccccgatggaatggtgctggcctacggctacgactcccaggcgcgattgaggtcggtgaccttgcagacagctgccggagcggtggcgtcggctatcgccgggtatgactatggcaatgcgcgcttcccggatgcactgaccacccaccgcgatgagcaggaccaggtgtatgcctcgtggacctacgatgcgcagcaacgcgtcgtacgcagcgtgcatggcgatccgaccggcaagatcgacgaggccactattgcctactctgggaacacttccacagtctccaatgcgctcggcaatgcagtaacgcgcactggcatctccaagcttggccaggccaaggttaccgccgtccagggcttgtgccccgaatgcacggttggggcgttcaaatcgcgcagctacgacggcaacggctaccccgaccaggaagtcgattttgcaggcgtcgccactgattggaggtacaacaaccgtggcttgttggccagcaagatcgaagcggacaacagcagcggcggccagaagcgcacgctgcagacggactggcaccccagtttccgtgtgcccaccgaccagcgcacctacgatgccagtgatattctggtcgcaaggacgagctggacctacaacagcaggggtcaggcactgaccgtatcgcggacggatccaagcggaggtcctacccgcgtcaccacccagcgctactgcgaagacagcgatactgctgccggcaattgttccttgcccggcttgctgttggccagcgatggcgcacgcacggatatcgcggatagcaccagctacacctactaccttgccgacgacgccagttgcacggcatcggcgagtacgtgcccgcatcgcaaaggcgaccgatggaaagtcaccaacgccctcggccaaaccaccgaatacctcgcctacgacggcgcaggccgtccgctgtccatcaaggacaccaatggcatcgtcaccgactacacctatcacccgcgcggttggctgaccgccagcaaggtgcgcggggccgatgcgtccagcgaggccgatgaccgcatcacccgcatcgactactggcccaccggcctggtcaggcaggtcacccagcccgatggcgcgttcactgccttcacctacgatgcggcgcatcggctgaccgatatcaccgacaacgcgggcaataccgtccactacacgctggacaacgccggcaatcgcgtcaaggaagacaccaaggacgcggccgccacgttgaagcgcacgctctcgcgcgtgtacaaccagctcggccagctcaagacccaggccaccgccgccagcgacccgaccgacttcgcgtacgacgccaacggcaacgccaccaaagtgaccgatgcgctggcaaccgcgacgcagagcgagtacgacccgctcaatcgcctctcgcacacgttgcaggatgtcgccggcatcaaggccgacacgaagttcgcctacgacgcactcgacaacctgaccaaggtcaccgaccccaaggggctggacaccacctacgactacaacggcttcggcgagttggtgaagctcaccagcccggacaccggcgtcaccagctacacctacgacagcgccggcaaccgcgccacccagaccgatgcgcgcggcaacaccaccgcctacagctatgacgcgctcaatcgcctgaccaaggtcacctaccccacctccagcctcgacgtcacctacacctacgacgtgacgcagaccgcttgcaccagcggcgaaacgttttcgatcggtcgcctgaccaagatgcaggacggcggcgccatcacccagtattgctacaaccgcttcggcgatctggtgcgcaaggtacagaccagcaacggcaccgcgttggtgctgcgttatgactacaccgtcggcggccagttgcgccgcatgacctatccggatggcgcagtcgtcgattacgtgcgcaatgcacaaggccagaccacgcaggtcggcgtgacgcccgccggcggtagccggcaggtgctgctgggcaatgccacttactacccgttcggcccggctgccggctggacctacggcaatggccgcaccctcgcccgccagtacgatctggactaccgcccgcaggccatccaggacacccgcccgggcgggctggacgtcggcttcggcttcgacccggccggcaacctcaccgcattgacgcccgcaggcaacaccacaccggagatcggcctgggctacgacgcgctgggccgcctgaccggcctcaccgatggtgtcaccggaactgtcattgacggctacagctatgacgccaccggcaaccgcctcagcaccaaggtcggcacggccacgcagatctacacctacccgaacgacagccaccgcctcagcgccgtcgccggcgtcgcacgtatatacgatgccaccggcaacaccaccgccatcggcggcaccgcccgccaatacacctatgacaccagcggccgcatgacccaggcccgccgcgccggcgcggtgaccatgaattatcgctacaacggtaaaggcgaacaggtgcgccgcttcctcggcaccaccaacacctacacgctctttgatgaagccggccactggctgggcgactacgacagcaacggcgcgcccaaacaacaggcgatctggctggacgatctaccggtcggcctgctggcgaatgccaacaaactgcactacatcgaacccgaccacctgggcagcccgcgcgtggtgatcgatccaacccgcgatgtggcggtgtggacgtggagcctgaagggcgaagccttcggcaacaccgcgccgaatcaggatccggatggggatggtgcggcggcggtgttggatatgcgcttcccggggcagcggtttgatgcggcgagtgggttgaatcaaaattatttccgggattatgaggcggctacagggcggtatgggcagagtgatccgattggaattgaaggtgggatcagcacatattcatacagtctaagctcgcctgttaaatatatcgatgttcttgggctggctgtctacctatgctcaaggccagtcaatgtcggctgggttccggattgggcgcgaccgtaccttcgccatatgtgggttaaaacatcctcgtttgaggctggaatgggaggtgaatgtcccattcctggacagggttgtgcagacgttccttactcggcaacagtgaccaaggatcactcgggccagtcgaaccaagcaggttctagctgtgagcttcagcaaaatgtggacgagcagtgcgtgaacgaaaaaataaaaccagggcaaccgacaggcacttggagcgtgttgaatcaatgccaaagcttctcaaacggcgttgtcaatcaatgtcgctacggccctcaaattggcccaatcttgccaaacacatttaggactcgcggccctcttggctcacaattttccccggagtag

SEQ ID NO: 55 - protein, 1510 aa, Xanthomonas
LDAMQDVQKRAPGYGSKWFGMLLAIVVTCVSLVPTIQIAHAYDSGVCNAGTRLCDQGQAYLNAIKEGNEAICIGGGINTHASLVLDLAYDQRYQAVVQCYRAFDNFTSESGLGESFYPLKCSARPTFTPAAGSTGNLCQEGCEYTVSNNGGQPVATPNGAVCSPPPPLDPGKNNGCCDGSTGPTTAGNPANVFTGTKLETAADYRAPAGHLEFVRYYSSAVGLQPTSALGNTWRHSYIRTITVTDASTLILTRPNGNFYTFKKSANGTWTPDWDVRETLSEVQDNGTLTGWQVTATDDSKEQFDLDGKLTGISYTDGEQLTLTYAGRQLQSVTDTRGRRLLFAYQADRITQVGLPDGMVLAYGYDSQARLRSVTLQTAAGAVASAIAGYDYGNARFPDALTTHRDEQDQVYASWTYDAQQRVVRSVHGDPTGKIDEATIAYSGNTSTVSNALGNAVTRTGISKLGQAKVTAVQGLCPECTVGAFKSRSYDGNGYPDQEVDFAGVATDWRYNNRGLLASKIEADNSSGGQKRTLQTDWHPSFRVPTDQRTYDASDILVARTSWTYNSRGQALTVSRTDPSGGPTRVTTQRYCEDSDTAAGNCSLPGLLLASDGARTDIADSTSYTYYLADDASCTASASTCPHRKGDRWKVTNALGQTTEYLAYDGAGRPLSIKDTNGIVTDYTYHPRGWLTASKVRGADASSEADDRITRIDYWPTGLVRQVTQPDGAFTAFTYDAAHRLTDITDNAGNTVHYTLDNAGNRVKEDTKDAAATLKRTLSRVYNQLGQLKTQATAASDPTDFAYDANGNATKVTDALATATQSEYDPLNRLSHTLQDVAGIKADTKFAYDALDNLTKVTDPKGLDTTYDYNGFGELVKLTSPDTGVTSYTYDSAGNRATQTDARGNTTAYSYDALNRLTKVTYPTSSLDVTYTYDVTQTACTSGETFSIGRLTKMQDGGAITQYCYNRFGDLVRKVQTSNGTALVLRYDYTVGGQLRRMTYPDGAVVDYVRNAQGQTTQVGVTPAGGSRQVLLGNATYYPFGPAAGWTYGNGRTLARQYDLDYRPQAIQDTRPGGLDVGFGFDPAGNLTALTPAGNTTPEIGLGYDALGRLTGLTDGVTGTVIDGYSYDATGNRLSTKVGTATQIYTYPNDSHRLSAVAGVARIYDATGNTTAIGGTARQYTYDTSGRMTQARRAGAVTMNYRYNGKGEQVRRFLGTTNTYTLFDEAGHWLGDYDSNGAPKQQAIWLDDLPVGLLANANKLHYIEPDHLGSPRVVIDPTRDVAVWTWSLKGEAFGNTAPNQDPDGDGAAAVLDMRFPGQRFDAASGLNQNYFRDYEAATGRYGQSDPIGIEGGISTYSYSLSSPVKYIDVLGLAVYLCSRPVNVGWVPDWARPYLRHMWVKTSSFEAGMGGECPIPGQGCADVPYSATVTKDHSGQSNQAGSSCELQQNVDEQCVNEKIKPGQPTGTWSVLNQCQSFSNGVVNQCRYGPQIGPILPNTFRTRGPLGSQFSPE

SEQ ID NO: 56 - DNA, 222 bp, Xanthomonas
atgcacgacgaacccggctactggatgtccgaaggcacgcaataccacctgctacgcgtacgcgaccaactgcgtctgctggccaccttggccacaccgcgcacccgcgaggaaggcgccatgggcatgccggccattcccctgcagcagtgggcgcattgcctgcagcacttggctgagcaggtggatgatgtgctgggggatttgggcaccgcccggtaa

SEQ ID NO: 57 - protein, 73 aa, Xanthomonas
MHDEPGYWMSEGTQYHLLRVRDQLRLLATLATPRTREEGAMGMPAIPLQQWAHCLQHLAEQVDDVLGDLGTAR

SEQ ID NO: 58 - DNA, 1461 bp, Xanthomonas
atgctcgcacttgcacagggcgcgtgggagtggctggccaccattccccacctcgaagcgctcctcctcgccgcctatgtgctgtacctgctatggatcgccggctggatcgtgctgcagaagcgcgaaccggtggccacgctgagctgggtgttgtcgctggcggcgctgccgtacctggggctgctgatctactactggctcgggccgcaaaaggtcaaacggcagcggctgcggcgcgggcgctcgcgctcgggcatggagagctacagcagcgtctgcccgccggatgccgattgcaccgagctggccaagatcgcccagtccaccaccgggctagcgcccagcagcgccaccgaagtgcactggctggtggatggcgctgccacgtacgccgccatcatcgaagcgatccggggcgcacgcgaccatatccatctggagtactacatcttccagcccgaccacagcggcaccgcgatctgcgctgcgctgatggaacgtgcccgtgccggggtcaaggtgcggctgctgatggatgcgatcggctcatcggcaatgacccgacgcgccttgcgcacgctgcgcgaggccggggtggaaaccgcctggttccacccctcgcagttgctcaagccattcaagcggccgtggttgaacctgcgcacccaccgcaaggtgatcgtcatcgatggccgcatcggtttcaccggtggcatcaacgtcaccgacgacgagaacgagcaggtgcgcaagcaggcgtatcgcgatctgcacgtgcgcctgcaaggccatgtagtgcgcagtctgcagctggtgttcctggaggattggctctatgccaccagccagggccgcgcagctttccacggccagcagctatggcccgacgatatgcccacccgcgcgcaaggcacggtcgatgcgcaggtgctggtgtcgggtccggattcgtcgtgggaagcgatccaccgtctgatggtcgccgccatccatgaagccaagcaccgcgtgtggctggtgactccatacttcgtgcccggcgaggcggcgcgtatggcgctgacctcggccgcgctcggcgggctggacgtgcgcctgttggtgccgcgggtcagcgattcgcgcctggtcacctacgcggcacgctcgtatttcgacgagctgctggaagccggtgtccgcatctacgagtacgggccgcgcatgctgcacaccaaggcgctgctggccgacgacgatgtctgcatcgtcggcagcgccaatttcgacagccgcagcttccggctgaatttcgagctgtcgatgctgttccgcgaccaggccgtggcggcagagatggccgggctgatcggcaccgatctgcaacaggcgcaggaagtgcaattcgcgcgacaccggccgttatggcgctcgcgcctgcccgaagcgtttgcccggctgctgtcgccgctgctctga

SEQ ID NO: 59 - protein, 486 aa, Xanthomonas
MLALAQGAWEWLATIPHLEALLLAAYVLYLLWIAGWIVLQKREPVATLSWVLSLAALPYLGLLIYYWLGPQKVKRQRLRRGRSRSGMESYSSVCPPDADCTELAKIAQSTTGLAPSSATEVHWLVDGAATYAAIIEAIRGARDHIHLEYYIFQPDHSGTAICAALMERARAGVKVRLLMDAIGSSAMTRRALRTLREAGVETAWFHPSQLLKPFKRPWLNLRTHRKVIVIDGRIGFTGGINVTDDENEQVRKQAYRDLHVRLQGHVVRSLQLVFLEDWLYATSQGRAAFHGQQLWPDDMPTRAQGTVDAQVLVSGPDSSWEAIHRLMVAAIHEAKHRVWLVTPYFVPGEAARMALTSAALGGLDVRLLVPRVSDSRLVTYAARSYFDELLEAGVRIYEYGPRMLHTKALLADDDVCIVGSANFDSRSFRLNFELSMLFRDQAVAAEMAGLIGTDLQQAQEVQFARHRPLWRSRLPEAFARLLSPLL

SEQ ID NO: 60 - DNA, 3291 bp, Xanthomonas
atggatcccattcgttcgcgcacaccaagtcctgcccgcgagcttctgcccggaccccaacccgatggggttcagccgactgcagatcgtggggtgtctccgcctgccggcggccccctggatggcttgcccgctcggcggacgatctcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggccgccgcgcgccaagccggcgccgcgacgacgtgctgcgcaaccctccgacgcttcgccggccgcgcaggtggatctacgcacgctcggctacagccagcagcaacaggagaagatcaaaccgaaggttcgttcgacagtggcgcagcaccacgaggcactggtcggccatgggtttacacacgcgcacatcgttgcgctcagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgcccccctgaacctgaccccggagcaggtggtggccatcgccagcaatattggtggcaagcaggcgctggagacggtgcaggcgctgttgccggtgctgtgccaggcccatggcctgaccccggagcaggtggtggccatcgccagccacgatggcggcaagcaggcgctggagacggtgcagcggctgttgccggtgctgtgccaggcccatggcctgaccccggagcaggtggtggccatcgccagcaatattggtggcaagcaggcgctggagacggtgcagcggctgttgccggtgctgtgccaggcccatggcctgaccccggagcaggtggtggccatcgccagccacgatggcggcaagcaggcgctggagacggtgcagcggctgttgccggtgctgtgccaggcccatggcctgaccccggagcaggtggtggccatcgccagcaatattggtggcaagcaggcgctggagacggtgcagcggctgttgccggtgctgtgccaggcccatggcctgaccccggagcaggtggtggccatcgccagcaatggcggtggcaagcaggcgctggagacggtgcagcggctgttgccggtgctgtgccaggcccatggcctgaccccggagcaggtggtggccatcgccagccacgatggcggcaagcaggcgctggagacggtgcagcggctgttgccggtgctgtgccaggcccatggcctgaccccggaccaggtggtggccatcgccagcaatggcggtggcaagcaggcgctggagacggtgcagcggctgttgccggtgctgtgccaggcccatggcctgaccccggaccaggtggtggccatcgccagcaatggcggtggcaagcaggcgctggagacggtgcagcggctgttgccggtgctgtgccaggcccatggcctgaccccggaccaggtggtggccatcgccagcaatggcggtggcaagcaggcgctggagacggtgcagcggctgttgccggtgctgtgccaggcccatggcctgaccccggaccaggtggtggccatcgccagcaatattggtggcaagcaggcgctggagacggtgcagcggctgttgccggtgctgtgccaggcccatggcctgaccccggagcaggtggtggccatcgccagcaatattggtggcaagcaggcgctggagacggtgcagcggctgttgccggtgctgtgccaggcccatggcctgaccccggagcaggtggtggccatcgccagcaatattggtggcaagcaggcgctggagacggtgcagcggctgttgccggtgctgtgccaggcccatggcctgaccccggagcaggtggtggccatcgccagcaatattggtggcaagcaggcgctggagacggtgcagcggctgttgccggtgctgtgccaggcccatggcctgaccccggagcaggtggtggccatcgccagccacgatggcggcaagcaggcgctggagacggtgcagcggctgttgccggtgctgtgccaggcccatggcctgaccccggagcaggtggtggccatcgccagcaatggcggcggcaggccggcgctggagagcattgttgcccagttatctcgccctgatccggcgttggccgcgttgaccaacgaccacctcgtcgccttggcctgcctcggcggacgtcctgcgctggatgcagtgaaaaagggattgccgcacgcgccggccttgatcaaaagaaccaatcgccgtattcccgaacgcacatcccatcgcgttgccgaccacgcgcaagtggttcgcgtgctgggttttttccagtgccactcccacccagcgcaagcatttgatgacgccatgacgcagttcgggatgagcaggcacgggttgttacagctctttcgcagagtgggcgtcaccgaactcgaagcccgcagtggaacgctccccccagcctcgcagcgttgggaccgtatcctccaggcatcagggatgaaaagggccaaaccgtcccctacttcaactcaaacgccggaccaggcgtctttgcatgcattcgccgattcgctggagcgtgaccttgatgcgcccagcccaacgcacgagggagatcagaggcgggcaagcagccgtaaacggtcccgatcggatcgtgctgtcaccggtccctccgcacagcaatcgttcgaggtgcgcgttcccgaacagcgcgatgcgctgcatttgcccctcagttggagggtaaaacgcccgcgtaccagtatcgggggcggcctcccggatcctggtacgcccacggctgccgacctggcagcgtccagcaccgtgatgcgggaacaagatgaggaccccttcgcaggggcagcggatgatttcccggcattcaacgaagaggagctcgcatggttgatggagctattgcctcagtga

SEQ ID NO: 61 - protein, 1096 aa, Xanthomonas
MDPIRSRTPSPARELLPGPQPDGVQPTADRGVSPPAGGPLDGLPARRTISRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPTHEGDQRRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 62 - DNA, 627 bp, Xanthomonas
gtggacgaagcgtgcgccaccatgccgaaccgacacacaaggtgcgacatggccaagccaacgttgttcttcctgcatgccctcggcgccagccggcatgagtggagccatgtcatcgcacagttaggtacccgcttcgactgtattgccctggacattcctggattcggcgatgcgcctgcgctgagccactgcaacacgaacgcgcttgttgactggtttagcgctgaagtcatcaagcggcaaccggcatgctggtttgcagtagggcacagcatgggcgggaagatcgccaccttgacggcggctcgggcgcgcgacggtgtggcgggccttgccggtttggcgggcgtggtgctcgttgccgcgtcgcccccggcacccgaaccgatgcaggagtcgcggcgccgcaccatgcttgcctggttcgaaacagggcagcctacgcgcatggaggctgcacattcgtggatgccaactgcgccgctccgctgcctgatgaacgcagggacgccgcgatcaacgacgttctgcgcaccgccgccagtgcatggatggcatggctcacccgcggtagccgcgaagactgcaccgcgcaggccgggtgcatcggcgtgccggcagtga

SEQ ID NO: 63 - protein, 208 aa, Xanthomonas
VDEACATMPNRHTRCDMAKPTLFFLHALGASRHEWSHVIAQLGTRFDCIALDIPGFGDAPALSHCNTNALVDWFSAEVIKRQPACWFAVGHSMGGKIATLTAARARDGVAGLAGLAGVVLVAASPPAPEPMQESRRRTMLAWFETGQPTRMEAAHSWMPTAPLRCLMNAGTPRSTTFCAPPPVHGWHGSPAVAAKTAPRRPGASACRQ

SEQ ID NO: 64 - DNA, 2550 bp, Xanthomonas
atggagtcgcgtctgtggcgactactggtgcacggcgtgacacccgagcagcgacaacgcctcgatgacttgctcaagcttgtcgaaggcagccggcagtcttggctggatcgattgcgcaaggggccggtacgcgtcagcgctccggcgctcgttgcggccttgctgcgaatcgaaaccgtgcgtggcttgggcattaagctgccaggcacccatgtgccgccgagccgcatcgcagcgctggcccgcttcgccagtactgccaaggtatccgccgtggctcgattgccggaggtgcgacgcatcgccacgctagtggccttcgtccactgcctggaagccagcgcgcaagacgatgccatcgatgtgctcgacctgctgctgcgcgagctgttcaccaaggctgagaaagaagatcgtaaggtcaggcagcgctccctcaaggatctggatcgggccgcctcgacgctggccgaggcatgccggatgctgctcgatccggccctgccggacggcgaactgcgcgagcgcgtctatgccgccatcggccacgatgaactggcccaggcgctcaatgaagtgcgcggtctggtgcgcccgcccaacgatgtgttctacaccgaactggaagcccgcaaggccaccgtctcgcgcttcctgccggcgttgctgcgcgtcatccgctttgacgccaatccggccgcgcaacctttggcgcaggcgttgcaatggctgcatgagaagcccgaccatgatccgcccacggccatcgtcggcaaggcgtggcaacgccatgtcgttcaggatgatggccgcatcaatgccacagcctattcgttctgcgcgctcgacaagctgcgcagtgcgattcgccgccgcgacgtgttcatcagcccgagttggcgctacgccgatccacgcgccgggctgctggccggagctgaatgggaggcctcgcggcctatcgtctgccgctcgctgagcctgtcggcgcagcccgaggccacgttgtccgagctgacgcgcgagctggacgaaacctaccgccgcgtcgccgcgcgcctgccccagaacgacgcagtgcgcttcgagaacgttggcgacaagacggaactggtgctcagtccgcttgaagcattggaggagccgccttcattgatcgcgctgcgcaacgaaatcaaggcgcgcatgccgcgcgtcgatcttccggaaatcctgctggaagtcgccggtcgtactggctgcatggaagcgttcacgcacctgactgaacgcaccgcgcgcgcggccgacctgaccaccagcctgtgcgcggtgctgatggccgaagcctgcaacaccggcccggaaccactggtgcggccagacactccggcgctcaagcgcgaccggctgatgtgggtcgatcagaactatgtgcgtgacgacacgctgacagcctgcaatgccgtgctggtggccgcgcaaagtcgtatcgcactggcgcgaacctggggaggtggcgatgtggcttcggccgacggcatgcgattcgtggtgccggtgcgcacgatccacgctggaccgaacccaaagtatttcaatcgcgggcgcggcgtcacttggtacaacttgctttccgatcagcgcaccggactgaacgcgatcactgtgcctggcacgctgcgcgacagtttgattttgctagcggttgtgctggagcagcaaacggagttgcagccgacccagatcatgaccgacaccggcgcgtacagcgatttggtgttcggtttgttccgcctctccaactatcgattctgcccgcgcctggccgatgttggcggtacccgtttctggcgcgtcgatcccgacgccgactatggcgacctcaatgcgctggcccggcagcgtgtgaatctcgaccgtatcaccccgcattgggatgatgtgctgcgtctggtcggctcgctcaagctcggtctggttccggcgatgggcatcatgcgcaccttacaggtcgatgaacggcccaccagcctggcgcaagccatcgccgaaatcggccgtatcgacaagaccatccacacgttgaatttcatcgacgacgaagcccgccgtcgcgccacgctgctgcaactgaatctcggtgaaggccgccacagcctggcgcgcgaggttttccacggcaagcgcggcgagctgttccagcgctaccgcgaagggcaggaagaccagttgagcgcgctcggcctggtcgtgaacatgatcgtgctttggaacacgctgtacatggatgcggtgctgacgcagttgcgcagcgaaggctaccccgtgaagccagaagacgaggcacggctgtcgccgttcggccacgagcacatcaacatgctcggacgctattcgttctcggtgccggaagctgtcgcgcgcggcgagccaagccgccgaaattcttggcttggtcatgcggcactggaacacgattgcatcagaattgttccgcactctggagaaagacgatgtctaccttcccgtgctactcgaagatgcggatggggccgtacacggtaa

SEQ ID NO: 65 - protein, 849 aa, Xanthomonas
MESRLWRLLVHGVTPEQRQRLDDLLKLVEGSRQSWLDRLRKGPVRVSAPALVAALLRIETVRGLGIKLPGTHVPPSRIAALARFASTAKVSAVARLPEVRRIATLVAFVHCLEASAQDDAIDVLDLLLRELFTKAEKEDRKVRQRSLKDLDRAASTLAEACRMLLDPALPDGELRERVYAAIGHDELAQALNEVRGLVRPPNDVFYTELEARKATVSRFLPALLRVIRFDANPAAQPLAQALQWLHEKPDHDPPTAIVGKAWQRHVVQDDGRINATAYSFCALDKLRSAIRRRDVFISPSWRYADPRAGLLAGAEWEASRPIVCRSLSLSAQPEATLSELTRELDETYRRVAARLPQNDAVRFENVGDKTELVLSPLEALEEPPSLIALRNEIKARMPRVDLPEILLEVAGRTGCMEAFTHLTERTARAADLTTSLCAVLMAEACNTGPEPLVRPDTPALKRDRLMWVDQNYVRDDTLTACNAVLVAAQSRIALARTWGGGDVASADGMRFVVPVRTIHAGPNPKYFNRGRGVTWYNLLSDQRTGLNAITVPGTLRDSLILLAVVLEQQTELQPTQIMTDTGAYSDLVFGLFRLSNYRFCPRLADVGGTRFWRVDPDADYGDLNALARQRVNLDRITPHWDDVLRLVGSLKLGLVPAMGIMRTLQVDERPTSLAQAIAEIGRIDKTIHTLNFIDDEARRRATLLQLNLGEGRHSLAREVFHGKRGELFQRYREGQEDQLSALGLVVNMIVLWNTLYMDAVLTQLRSEGYPVKPEDEARLSPFGHEHINMLGRYSFSVPEAVARGEPSRRNSWLGHAALEHDCIRIVPHSGERRCLPSRATRRCGWGRTR

SEQ ID NO: 66 - DNA, 660 bp, Xanthomonas
gtgacgggcaagcccgtacgcgcacgcagcaaaaccaacgtcgcgtcgtcgcgggttcacgcaatgggttgcgtacagcaggcaccaaccagacatccctcgccgaagccgccgccgcccggacatgaccatgcgcacgcgggccatggcatgtcgatgatgtgcaatggcgggttggtcgctagcatgcagagacgttctctgcccaagcgagtgcccatgtccaccgtcacgctccaccgcgtcatacgtgccagccccgagcggatctatcgcgccttccttcaggccgatgcggtggcaaagtggctgccgcccaatggcttcacctgcaccgtggagcatttgcatgcgcatgttggcggcaccttccgcatggcgttcaccaatttcgccaatggcgagcggcacgcgttcggcggcacctaccgcgagctgcaacccaacgtgctgttgcgctacgacgatgcgttcgacgacccctcgctgcctggcaccatggtcaccaaggtgcagttgcatacgctgccgtgcggtgccgatctgcacgtggtgcaggaaggcattcccgaacagattccgctgcatgcctgctacctgggttggcaggattcgttgatgctgttggcgcatctggtggaagccaatccggagcactag

SEQ ID NO: 67 - protein, 219 aa, Xanthomonas
VTGKPVRARSKTNVASSRVHAMGCVQQAPTRHPSPKPPPPGHDHAHAGHGMSMMCNGGLVASMQRRSLPKRVPMSTVTLHRVIRASPERIYRAFLQADAVAKWLPPNGFTCTVEHLHAHVGGTFRMAFTNFANGERHAFGGTYRELQPNVLLRYDDAFDDPSLPGTMVTKVQLHTLPCGADLHVVQEGIPEQIPLHACYLGWQDSLMLLAHLVEANPEH

SEQ ID NO: 68 - DNA, 1899 bp, Xanthomonas
atgccccccgtttccgccctcgatgccggcacgccaacgctgccgccggaagcgccgctggcgatgcccgaacagaacttgcgcgaaggtagcctgcaggtgcgccaccagcgcacttcgccgcccggcatcggcgtgcgccgcttctatttgatcggcggcaccttcgccaccaccgccatcgccgtatgggtgatgctcagcgtgctgtggcccgacggcatcagcgtgctggaaggctgtctgctcggcctgttcgtgctgctgttcgcctggatcgccatgtccttcgccagcgccgtggctggcttcgtcaccgtggtggcgcgcgccggccgcaagctcggcatcgacccggagcaacccctgcccacgctgcgctcgcgcaccgcgttgctgatgcccacctacaacgaagacccgcgccggctgctggctggcctgcaggccatctacgaatcggtcgccgagaccggccagctggagcatttcgatttcttcgtgctcagcgacaccacccgcgagcacatcggccgcgccgaagagctggtctacaacgcgttgtgcgaccgcgtcggcgggcacggccgcattttctaccgacgccgtgccgacaacgccgcgcgcaaggccggcaacgtggccgactgggtgcgccgcttcggcggcagctacccgcagatgctgatcctggacgccgacagcgtgatgaccggcgacaccatcgtgcggctggtcgcagggatggaaaacaacccggacgtgggcttgatccagaccttgccggcagtggtcaacgggcagaccctgttcgcccgcatgcagcagttcggcggccgcgtgtacgggccgatcatcgccttcggtgtggcctggtggcacggcgccgagagcaactactggggccacaacgccatcatccgcacccaggccttcgccgatcacgccggcctgccgtcgctgcgcgggcgcaagccgttcggcgggcatgtgctcagccacgacttcgtggaagcggcgctgatgcgccgcggcggctgggccatgcacatggtgccgtacctgcagggcagctacgaagaaggcccgcccacgctgaccgacctgctgatccgcgaccgccgctggtgccagggcaacctgcagcacgccaaggtggtcggcgccaagggcctgcactggatcagccgcatgcacatgctgatcggcatcgggcattacttcaccgccccgatgtggggtctgttgatgctgatcggcatcggcattccgctggccggcggcgatatcgatctggccggcgacctgccgttctcgcccgcccgttactggcatggcagcagccagggcaatgcgatctggatcttcgtctgcaccatgttcgtgttgctggcgcccaagctgctcggttacatcgcgttgctgctcaacccgcgcgaactgcgcgcctgcggcggtgccttccgcgctgcggtgagcatcctgctggaaacggtgctggccgccttgatggcgccggtggtgatgtacctgcagtcgcgcggcgtgttcgaggtgctggcgggcaaggattcgggctgggatgcgcaggtgcgcgacgacggcaagctgtcgtggccggcgttgttgcgcagctatggcggtctgaccgtgttcggcctgttcatgggtgcggtggcctatgcggtctcgccggcgctggccgcgtggatggggccggtgatcgttggcatggcattgtcgatcccggtggtggcgctgacctcgctgcgtcgcaccggcatggcgctgcgccgtgccggtattttctgcatccccgaagaactcgacccgcccaaggtgctggtgcgtgcgtccgagctgcgccgcgccgccgcgctggaaccgtcgctgatctga

SEQ ID NO: 69 - protein, 632 aa, Xanthomonas
MPPVSALDAGTPTLPPEAPLAMPEQNLREGSLQVRHQRTSPPGIGVRRFYLIGGTFATTAIAVWVMLSVLWPDGISVLEGCLLGLFVLLFAWIAMSFASAVAGFVTVVARAGRKLGIDPEQPLPTLRSRTALLMPTYNEDPRRLLAGLQAIYESVAETGQLEHFDFFVLSDTTREHIGRAEELVYNALCDRVGGHGRIFYRRRADNAARKAGNVADWVRRFGGSYPQMLILDADSVMTGDTIVRLVAGMENNPDVGLIQTLPAVVNGQTLFARMQQFGGRVYGPIIAFGVAWWHGAESNYWGHNAIIRTQAFADHAGLPSLRGRKPFGGHVLSHDFVEAALMRRGGWAMHMVPYLQGSYEEGPPTLTDLLIRDRRWCQGNLQHAKVVGAKGLHWISRMHMLIGIGHYFTAPMWGLLMLIGIGIPLAGGDIDLAGDLPFSPARYWHGSSQGNAIWIFVCTMFVLLAPKLLGYIALLLNPRELRACGGAFRAAVSILLETVLAALMAPVVMYLQSRGVFEVLAGKDSGWDAQVRDDGKLSWPALLRSYGGLTVFGLFMGAVAYAVSPALAAWMGPVIVGMALSIPVVALTSLRRTGMALRRAGIFCIPEELDPPKVLVRASELRRAAALEPSLI

SEQ ID NO: 70 - DNA, 411 bp, Xanthomonas
atgaagtttgaaatcgataacaagacgatcgccgatgccaaaatggtcggcacccaggcagtaaaggcattgctattcatttcgttgctgatggtggccggcggtgcatccgcagacggcaatggtcttgcagacaccgacacccgcgtgtgtggctttttgaataatgtgaccaagctgttgaacatgggctcgatcgcagtggtcaccatcgcagtcatcgtggccggctaccagatcgctttcgcacacaagcgtatctccgaggtctcgccaattctcattggtggcgttctcatcggtgctgctggccagatcgccaacatgatccttcagaataaaaacggcagttcgggtgattgctcgacgatcaccggttcactgatgcagatcgctcaatactatgcataa

SEQ ID NO: 71 - protein, 136 aa, Xanthomonas
MKFEIDNKTIADAKMVGTQAVKALLFISLLMVAGGASADGNGLADTDTRVCGFLNNVTKLLNMGSIAVVTIAVIVAGYQIAFAHKRISEVSPILIGGVLIGAAGQIANMILQNKNGSSGDCSTITGSLMQIAQYYA

SEQ ID NO: 72 - DNA, 405 bp, Xanthomonas
atgtccaaatcaaccctagattccattgctacggccaaagccaagcttgcagaagagctgagaaagctggaagagcaagaagccaatctgttggaggaagaggcatcgaacgcttttcagcaagcctcagatctgctgacccgcttcggccagcactttaccgccaagcagaaagcagatcttgcagctcaactcggggcttctgttggcggatctggccgcccgaagaaagcagccggcactcgcaaggaagtggccccgaagtattggcttccccacacgcaagaaacttggtctggtcggggtaggactccacgcgcttttgcagcttacgaaggatcggcggcttataaagaatggaaggcaaagcatccggatgaaaaatttccgaagtttccaggataa

SEQ ID NO: 73 - protein, 134 aa, Xanthomonas
MSKSTLDSIATAKAKLAEELRKLEEQEANLLEEEASNAFQQASDLLTRFGQHFTAKQKADLAAQLGASVGGSGRPKKAAGTRKEVAPKYWLPHTQETWSGRGRTPRAFAAYEGSAAYKEWKAKHPDEKFPKFPG

SEQ ID NO: 74 - DNA, 1437 bp, Xanthomonas
atgttcagcgccgaggtggaacccgcgtggagttttgaagcatggcgtgcgttggcgcgggcaggctggtgcgcgcaggtggagccggacgatgtggcctggaatggcggggcgcagggcggcttgttgatggggcagggactgttggcgatgccggccgtggtgccaccaccgcgggtctctgcggatttcatgcaactggcggcctcggtgctgtgtcaccgcgacccgcaacggcacgccgtgttgtatcgcttgctgtggcgggttgcctctggggaaaaagccctgctggaacgcgccaccgatgtggatgtgcaccggctacggcaatggcaaaaagcggtgcagcgcgatacccacaaaatgaaggccttcgtgcgctttcgtcgcctgcccggggaagaagaagacttcatgtcctggttcgagcccgagcactggatcgtggatcgcgtggcgccgttttttgcgcgccggtttgccggcatgcgttgggcgatcctgaccccgtaccgcagtgtgcgttgggatgggcaggcgttgaccttcggcgaaggtgcggtgcgcacccaggtacccgccgacgatgcgcaggagacgctctggcgcacgtactacgcgcatatcttcaacccggcgcggctcaacccgaccatgatgcggcaggaaatgccgcagaaatactggaagaacttgcccgaagccacgctgttgccggagctgatccgcgatgccggcatgcgtgttcgcgagatggccgagcgcgcgcccgagccggtacggcgacgggtgccggcagcgcctgcgccattgccggaagcggcaacccaaagcctggcgcagttgcgcactgccgcacgcgattgccggcgttgcgaactgtggcagccggcaacgcaaacggtgttcggcgaaggatccgacgatgcgtcggtcatggtgatcggcgagcagccgggcgatgaagaggacttgagcggtcgtccgtttgtcgggcccgccgggcgcctgttcaacatggcgttaggcgagctgggtgtggaccgcgagacgttctacgtcaccaatgcggtcaagcattttcgtttcgagcaacgcggcaaacggcggctgcatcgcaatccggagcgcacccatgtgcaggcctgcagcgggtggttgcaggccgaacgggcgcacttgcgtccggcgcagatcgtctgcctgggcgcgactgcggcgcaagccgtgttgggaagccgcttccgattgatgcagcaacgcgggcaatggcagcgcctggacgacggcacgcctgtattgaccacggtgcacccgtcctgggtattgcgccagccatcgacggatgcgcgcgaagaagggtatcgcggatttgtcgaggatctccgtcagttgctggatccaccgccgccgtccagcgatcagtcgagatag

SEQ ID NO: 75 - protein, 478 aa, Xanthmonas
MFSAEVEPAWSFEAWRALARAGWCAQVEPDDVAWNGGAQGGLLMGQGLLAMPAVVPPPRVSADFMQLAASVLCHRDPQRHAVLYRLLWRVASGEKALLERATDVDVHRLRQWQKAVQRDTHKMKAFVRFRRLPGEEEDFMSWFEPEHWIVDRVAPFFARRFAGMRWAILTPYRSVRWDGQALTFGEGAVRTQVPADDAQETLWRTYYAHIFNPARLNPTMMRQEMPQKYWKNLPEATLLPELIRDAGMRVREMAERAPEPVRRRVPAAPAPLPEAATQSLAQLRTAARDCRRCELWQPATQTVFGEGSDDASVMVIGEQPGDEEDLSGRPFVGPAGRLFNMALGELGVDRETFYVTNAVKHFRFEQRGKRRLHRNPERTHVQACSGWLQAERAHLRPAQIVCLGATAAQAVLGSRFRLMQQRGQWQRLDDGTPVLTTVHPSWVLRQPSTDAREEGYRGFVEDLRQLLDPPPPSSDQSR

SEQ ID NO: 76 - DNA, 3195 bp, Xanthomonas
atgctgaccaacgtcatccggtttgcaattgcgcaacgttggttgatgctggcgttgacaggggtgctgattgcgatcggggcgtggagtttttcgcgcttgccgatcgatgcaacacccgacatcaccaacgtgcaggtgcaggtcaataccgcagcgccgggctattcgccgctggaatccgagcagaggataacgtttccgttggagacggtgctggcgggcttgccgggtctggaatccacgcgctcgctgtcgcgctatggcttgtcgcaggtcaccgcggtcttcgccgatggcaccgatctgtatttcgcgcggcaacaggtggccgaacgtctgcagcaggtcaagtcgcaattgcccgccgatctggagccgcagctgggaccgatcgctaccggcctgggcgagatcttcatgtacacggtcgaggccaagccgaatgcgcgcaagcccgatggcagtgcatggacggcgaccgatctgcgcaccttgcaggattgggtggtgcgcccgcagctacgcaacgtgccgggcgtcaccgaggtcaacaccatcggcgggtatgcgcggcagatccatatcacgcccgatccggcgcgcctggtcgcgttgggatttaccctggacgacgtcgcccgagcggtcgagtccaacaaccgcaacatcggcgccggctatatcgaacgtaacggccaacagtttctggtgcgggtgccggggcaggtggacgacatcgcgcagatcggcgcggttgtgctggatcggcgcgccggcgtgccgatccgcgtgcgcgatgtggcgcaggttggcgaggggcgcgagttgcgcaccggcgccgccacccaggacgggagcgaagtggtgctcggcacggtcttcatgctcgtcggtgccaatagtcgcaccgtggcgcaggccgctgcgcagcggctggaagtggccaacgccagcttgccggccggtgtgcaggcggtgccggtgtacgaccgcaccgcgttggtggaccggaccatcgtcaccgtcgccaagaacctgatcgaaggcgcgttgctggtgatcgtggtgttgttcctgttgctgggcaatgtgcgcgcagcgttgatcacggctgctgtgattccgttggcgatgctgttcacgctcaccggcatggtgcgtggtggcgtgtccggcaatctgatgagtcttggcgcgctggatttcggtttgatcgtcgatggcgcggtgatcatcgtggagaactgcctgcgccgctttggcgacgcgcaacgtcggctggggcgcgtactcgaacgcgacgagcgtttcgagctcactgccgaagccactgccgaggtgatccgccccagcctgttcggcctgggcatcatcactgcggtgtatctgccggtgttcgcgctcaccggtatcgaaggcaagatgttccacccgatggcgatcaccgtggtgctggcactgaccggcgcgatgctgctgtcgctgaccttcgtgcctgcagcgattgcgttgctgctcggcggcaaggtggccgagcacgaaaaccgcgcgatgcgctgggcgcgcggcgtctatgcgccattgctcgatcgggcgcttcggcacggccgctgggtggccattgcagcgcttgcgacggtggcgctgtgcgcggtattggccacgcgtctggggagcgagttcatccccaacctggacgaaggcgatgttgcgctgcatgccttgcgcattcccggcaccagcctggaacaggccatcaccatgcaatccacgctggaaaagcgcatcaagcagtttccggaggtggcgcatgtgttcggcaagctcggtactgccgaagtggccaccgacccgatgccgccatcggtcgccgacacgttcctgatcatgcatccgcgcgcgcagtggccggatccgcgcaagcgcaaggcgcagttgctggccgaaatcgaggaggcggtgaagcagttgccgggaaacaattacgagttcacccagccgatccagatgcgcatgaacgagctgatttccggcgtgcgtgcggatgtggcgatcaaggtgtacggcgacgatctggacacgctggtcaggctcgggcagcgcgtgcaggaaatcgccagcgcggtgccgggcgcggccgatgtgagcctggaacaggccaccggcctgccgatgctggcggtggtacccgatcgcgccgcgctggccggctacgggctcaatcccggtgtggtgcaggacaccgtggccgcggcagtgggcggacaggaggccgggcaattgttcgaaggcgaccggcgcttcgacatcgtggtgcgcctgccggaatctctgcgccaggaccccactgcgctggccgatctgccgatcccgctgcgtggcgatggcgagcgtgccgatgcggacgagtccagccgcgcggcaggctggcgcagtggcgagccgatcaccgtgccgctgcgcgaggtcgccaagatcgacaccgtgctggggcccaaccagatcaaccgcgaagacggcaagcgccgcatcgtgatcaccgcgaacgtgcgcgatcgcgatctggggagcttcgtggccgaggtgcggcagcgcgtgcagacgcaggtcaagctgcccaccgggtactggatcggctacggcggcaccttcgagcagctgatctccgccagccagcgcctggcctgggtggtgccgggcaccttggtgttgatcttcgcgctgctgtattggtccttcggctcgctgcgcgatgcgctggtggtgttcagcggcgtgccgctggcgctgaccggcggcgtgctggccttggccctgcgcgggctggccttgtcgatttccgccggcgtgggctttattgcgttgtcgggcgtggcggtgctcaacggcctggtgatgatcgcgttcgtgcgcagcctgcgcgctgacggcatgccgctggagcgggcattgcgcgaaggcgcgctggcgcggttgcgaccggtgctgatgaccgccctggtggccgcgctgggctttgtgccgatggccttcaacgtcggtgccggtgccgaagtgcagcgcccgctggccacggtggtgatcggcggcatcgtctcctccaccctgctcaccctgctggtgttgccggtgctgtatcgctggttgcatcggcagcgggcgccccgtcgggagcgtggagcgcctgagtag

SEQ ID NO: 77 - protein, 1064 aa, Xanthomonas
MLTNVIRFAIAQRWLMLALTGVLIAIGAWSFSRLPIDATPDITNVQVQVNTAAPGYSPLESEQRITFPLETVLAGLPGLESTRSLSRYGLSQVTAVFADGTDLYFARQQVAERLQQVKSQLPADLEPQLGPIATGLGEIFMYTVEAKPNARKPDGSAWTATDLRTLQDWVVRPQLRNVPGVTEVNTIGGYARQIHITPDPARLVALGFTLDDVARAVESNNRNIGAGYIERNGQQFLVRVPGQVDDIAQIGAVVLDRRAGVPIRVRDVAQVGEGRELRTGAATQDGSEVVLGTVFMLVGANSRTVAQAAAQRLEVANASLPAGVQAVPVYDRTALVDRTIVTVAKNLIEGALLVIVVLFLLLGNVRAALITAAVIPLAMLFTLTGMVRGGVSGNLMSLGALDFGLIVDGAVIIVENCLRRFGDAQRRLGRVLERDERFELTAEATAEVIRPSLFGLGIITAVYLPVFALTGIEGKMFHPMAITVVLALTGAMLLSLTFVPAAIALLLGGKVAEHENRAMRWARGVYAPLLDRALRHGRWVAIAALATVALCAVLATRLGSEFIPNLDEGDVALHALRIPGTSLEQAITMQSTLEKRIKQFPEVAHVFGKLGTAEVATDPMPPSVADTFLIMHPRAQWPDPRKRKAQLLAEIEEAVKQLPGNNYEFTQPIQMRMNELISGVRADVAIKVYGDDLDTLVRLGQRVQEIASAVPGAADVSLEQATGLPMLAVVPDRAALAGYGLNPGVVQDTVAAAVGGQEAGQLFEGDRRFDIVVRLPESLRQDPTALADLPIPLRGDGERADADESSRAAGWRSGEPITVPLREVAKIDTVLGPNQINREDGKRRIVITANVRDRDLGSFVAEVRQRVQTQVKLPTGYWIGYGGTFEQLISASQRLAWVVPGTLVLIFALLYWSFGSLRDALVVFSGVPLALTGGVLALALRGLALSISAGVGFIALSGVAVLNGLVMIAFVRSLRADGMPLERALREGALARLRPVLMTALVAALGFVPMAFNVGAGAEVQRPLATVVIGGIVSSTLLTLLVLPVLYRWLHRQRAPRRERGAPE

SEQ ID NO: 78 - DNA, 1278 bp, Xanthomonas
atgatcatgccttcgcgccttttgcgtttaacgctcggtgtcagcgtctgtgtggccgcaaccagcgtagcagcgcaagccatcgctcccgaaacaaccgcatcctccgcaaccgacgccggtgggcaacagcaggatcccgcacccactgcgagcttcgaacagtggctggcagacttccgccaacgcgcgcttgccgccggcattggagccaccacgctggataacgcattggctggtgtcacacccgatccagcggtgcatgaactggatcagcggcaacccgaattcacccagtatctatgggactatctcgacgcgcgtgtcacaccttcggccatccaggaaggacagcagctgctgatcagccagcacgccctcttcgagaagttgcggcagcactacggtgtggatccgggcattctgaccgccatctggagcatggaaagcgggtacggcaagcagatcggggacttttatgtcattcgctccctggcgacactggcccatgagggacggcgcaccacgtatggcaacacgcagctgcttgcggccctgcagatcctgcagacggaaaaaagcattgatcgctcgcaactggtgggttcgtgggccggcgccatgggacagacgcaattcattccgagcacctatcgcgactacgccgtcgacgaggatggcgaccagaagcgggatgtctggaattccaaggcagacgctttgggctctgcggcgaattatctcaagcagaacaattggaccagcgctgttccctggggccaggaagtccagctgtcagccggcttcgactacgcgcaggccgacctgacgatcaagaagaccgtcgccgaatggcagcgcctgggcgtggcgccccgcaggccgattgcgcccgcactggcgcagcagttggcatcggtgttgttgcccaccggataccgcggcccggccttcctagtgttcgataacttccgcagcattctgcgctacaacaattccaccgcctatgccctggcggtcggcttgctggcagacggctatgcgggcagggccggggtcaagcaaccctggcccaaagacgatccgccgctcaatagcacggcccagatcaccgagctgcagcagaggctgaccgacaagggattcgacgtgggcggcatcgacggtgtgctgggggcgcaaactcgccagggaatccgtgcattccagcgtagccagcaattgccgcaggacggttacgccagtacgtcgctgctggcccgtctgcgggccgcctga

SEQ ID NO: 79 - protein, 425 aa, Xanthomonas
MIMPSRLLRLTLGVSVCVAATSVAAQAIAPETTASSATDAGGQQQDPAPTASFEQWLADFRQRALAAGIGATTLDNALAGVTPDPAVHELDQRQPEFTQYLWDYLDARVTPSAIQEGQQLLISQHALFEKLRQHYGVDPGILTAIWSMESGYGKQIGDFYVIRSLATLAHEGRRTTYGNTQLLAALQILQTEKSIDRSQLVGSWAGAMGQTQFIPSTYRDYAVDEDGDQKRDVWNSKADALGSAANYLKQNNWTSAVPWGQEVQLSAGFDYAQADLTIKKTVAEWQRLGVAPRRPIAPALAQQLASVLLPTGYRGPAFLVFDNFRSILRYNNSTAYALAVGLLADGYAGRAGVKQPWPKDDPPLNSTAQITELQQRLTDKGFDVGGIDGVLGAQTRQGIRAFQRSQQLPQDGYASTSLLARLRAA

SEQ ID NO: 80 - DNA, 345 bp, Xanthomonas
atggcacgcatgttgcgcgaatggccgccagacattgcagcgatcggcgaagctaagtcgccacatgtggcgcgcgatctccgcatgtcagagctgctatcggccagcaaccgcggcgagcagtcatctgcagaaatggaaggcgcagacgcctatatccgcgggttcgtggtgcagtcattccttcgaccaatcccaagcgtagtacgtgcgtgctgcgccggagccgaacatgttcgtccggccgtggtacggattctcggttgctccggtgcgtcgatgacttgcacaggccaatgtgcggtcgccaacgacagcggcctggcagagcgctggccgccgtga

SEQ ID NO: 81 - protein, 114 aa, Xanthomonas
MARMLREWPPDIAAIGEAKSPHVARDLRMSELLSASNRGEQSSAEMEGADAYIRGFVVQSFLRPIPSVVRACCAGAEHVRPAVVRILGCSGASMTCTGQCAVANDSGLAERWPP

SEQ ID NO: 82 - DNA, 537 bp, Xanthomonas
gtgcatgtcgcagatcggcatcccgacgggcagcgagcgtcatcgggtggctttcagttgctgcgggcatcgatggactacggccgcgcattgctcttcttgttagaaacccatcccatcgatctgccggccgtggcgttggggatgcatcgctcgcagatcgagcagttcctgcgagcggtgtttgtccagttcctcgccgacgatgatcagtttcaagactttcttgaggaggaccgaggcccccgcaagaagaatgaaaaaggaaagtgggtcgccatttcattaaaggacttggccgccgacgtcgaaacgaccattgcacgaataggtcaggacgaggagccccaaaagcttgcccgcaccgtaagcaatgtgtgggatccgctttgcgggttggtccatggcggacgggcatccggactatgtatcaagatccgaatggtcagatcggtgccaacgtgccggcaggcattttgtttcaggccacggtcaattcggtggcgacgacaaacctctgcgttatcactgccctga

SEQ ID NO: 83 - protein, 178 aa, Xanthomonas
VHVADRHPDGQRASSGGFQLLRASMDYGRALLFLLETHPIDLPAVALGMHRSQIEQFLRAVFVQFLADDDQFQDFLEEDRGPRKKNEKGKWVAISLKDLAADVETTIARIGQDEEPQKLARTVSNVWDPLCGLVHGGRASGLCIKIRMVRSVPTCRQAFCFRPRSIRWRRQTSALSLP

SEQ ID NO: 84 - DNA, 1230 bp, Xanthomonas
atgactagcactgcgcttccccaaattccagataacaagcttgcgcttgagatgcgtggagttaccaaggactacccagtctatgggcatcctctcaagcgtctattctcgttgttgcgcccatcgggtgatcgtgcatcccacaactttcgcgcgctggaggatataaccctccttgtaccaaggggcgaaaccgtcggcatcatcggtcggaacggtgccggaaaatcgacccttctacagatcctgtgcggcacgctttcgccgaccaagggcactgtccgcatacgggggcgttttgcggcactgctggagttgggggccggcttcaaccctgacttcagcggccgtgagaacgtctatctcagcgcgtctctgcttggcctgactcatgcggaagtcgatcaaagcctggacaagatcattgagttcgccgatatcggtgaattcatcgaccgcccggtcaagacctattccagcggcatgtacgtgcggctggctttttccgtcgccattcacacagagcctgacgtgctgatcatcgacgaggcactgtctgtgggtgatatccgctttcagataaagtgtctggcgcgaatcgagcagatccgcgcgcgtggagccaccgtcctgtttgtctcgcattcgctggagcaggtgaagcggttctgccaaaccgcgatctggctcgagcaggggcaggtgaaattgcatggcgaggcgagttttgtcgcggaccggtttcgggactacgaactgggacgactggcgcccccgtcaggcgaagcgaatctagttgcggaaaagtctcctgcaaaaagagattcggtccccgctcacatcgagcgtacgacgatgtctaccgacatgttggcgccctttgaacctttgacagtcgagatcgaatacgcggtcaatgacgacatgatcgatggcttgctcttgggcgttgcaataaaaggcctggatggtcagcacatctttggcccgaatacgcatcttgagcagatcgatattcctgctagccgtggaactcacggtattacgtaccacattccttccatgccgttgttaacaggcacatatcgcatcgatgtcggtctttttaccgataagggcctggtgtgcttggattacctgtcttcggtaaatgagttcaccgtcgctgcgccgtacttcagcgaggggctggtctatatttctcatgagtggcgtgtccatgggcgttga

SEQ ID NO: 85 - protein, 409 aa, Xanthomonas
MTSTALPQIPDNKLALEMRGVTKDYPVYGHPLKRLFSLLRPSGDRASHNFRALEDITLLVPRGETVGIIGRNGAGKSTLLQILCGTLSPTKGTVRIRGRFAALLELGAGFNPDFSGRENVYLSASLLGLTHAEVDQSLDKIIEFADIGEFIDRPVKTYSSGMYVRLAFSVAIHTEPDVLIIDEALSVGDIRFQIKCLARIEQIRARGATVLFVSHSLEQVKRFCQTAIWLEQGQVKLHGEASFVADRFRDYELGRLAPPSGEANLVAEKSPAKRDSVPAHIERTTMSTDMLAPFEPLTVEIEYAVNDDMIDGLLLGVAIKGLDGQHIFGPNTHLEQIDIPASRGTHGITYHIPSMPLLTGTYRIDVGLFTDKGLVCLDYLSSVNEFTVAAPYFSEGLVYISHEWRVHGR